(19) United States
(12) Patent Application Publication
(10) Pub. No.: US 2023/0368891 A1
(43) Pub. Date: Nov. 16, 2023

(54) COMPUTER-IMPLEMENTED SYSTEM AND METHODS FOR INTEGRATED CONTEXT-BASED PERSONAL DIET MANAGEMENT

(71) Applicant: Practice Innovations & Development, LLP, Rockville, MD (US)

(72) Inventors: John Oti, Tyler, TX (US); Christopher Oti, Rockville, MD (US); Richard Oyelewu, Berwyn, PA (US)

(73) Assignee: Practice Innovations & Development, LLP, Rockville, MD (US)

(21) Appl. No.: 18/318,088

(22) Filed: May 16, 2023

Related U.S. Application Data
(60) Provisional application No. 63/342,256, filed on May 16, 2022.

(51) Int. Cl. G16H 20/60; G06F 9/451

(52) U.S. Cl. CPC G16H 20/60; G06F 9/451

(57) ABSTRACT

Computer-implemented system for integrated context-based personal diet management preferably uses an internet-based client-server system architecture, in which a system server provides centralized food storage and user configuration services for a multitude of client devices. Through a client device a user can select from a choice of target diets, build a dish of food items, and carry out a context-sensitive calculation. The calculation results in a report that includes a comparative analysis of the dish against the dietary objectives of a target diet, with met and unmet dietary goals highlighted, and with needed corrective dish modifications made on how to improve the dish. Food items are sourced, from a native regional source available to all users, as well as from custom food sources that are available from food vendors such as restaurants, by scanning of unique scan codes displayed by vendor users.

### CROSS REFERENCE TO RELATED APPLICATIONS

This application claims priority to and the benefit of the filing date of U.S. Provisional Application No. 63/342,256, filed on May 16, 2022, entitled “COMPUTER BASED SYSTEM AND METHODS FOR INTEGRATED CONTEXT-BASED PERSONAL DIET MANAGEMENT”, which is hereby incorporated by reference in its entirety.

### FIELD OF THE INVENTION

There is a growing global awareness of the importance of a sound personal diet on the overall health and wellbeing of an individual. Much evidence of this can be found in the plethora of websites and software applications offering calorie-counting and diet-monitoring experiences. These services usually aim to assist their visitors and users with their weight-loss aspirations and/or their needs for healthy and balanced diets.

Frequently, a person's dietary needs are dictated by an underlying medical condition. Heretofore, to address such needs, an individual typically navigates a patchwork of disparate tools and resources to find the answers he or she needs.

Therefore, a need exists for novel systems and methods which enable the planning and management of personal medical diet goals.

### BRIEF SUMMARY OF THE INVENTION

A computer-implemented integrated context-based personal diet management system and methods are disclosed which provide an integrated, general-purpose system for the planning and management of personal medical diet goals. In preferred embodiments, the system provides the means for an individual to monitor and analyze the nutritional value of his or her food intake through a system of context-based inputs in a simple three-step process. The reliance on contexts leads to naturally-flowing user-experiences that mimic real life. The user of the system has instant access to food nutritional data from a variety of sources, such as an “always on” geographical food source, as well as custom food sources that are available from food vendors such as restaurants. The geographical source preferably represents the native food context for the system and presents a familiar food milieu to the user—it is preferably available to all users. The custom sources may be opportunistic and allow a user to gain access to food data by scanning a food menu code provided by the vendor. In preferred embodiments, the vendor is a special user of the system that has access to additional interaction tools for creating custom food content. Thus, a user may use the system to assist him or her in making educated food choices in real time and food vendors may facilitate the extension of the appliance's data so that a user has additional access to virtually an inexhaustible variety of custom-dish nutritional content.

In some embodiments, the system may include: a system database; a dietary client device having a display screen; program instructions stored in one or more computer-readable memories; and one or more processors configured to execute the program instructions to cause the system to perform operations that include: generating a graphical user interface (GUI) on the display screen of the dietary client device, in which the GUI displays data of a plurality of diet data records on the display screen of the dietary client device, and in which each diet data record of the plurality of diet data records is associated with a recommended nutritional objectives data record in the system database; receiving, via the dietary client device, user input selecting a diet corresponding to a diet data record of the plurality of diet data records that is to be identified as a target diet data record; detecting a geographic location of the dietary client device; displaying, via the GUI on the display screen of the dietary client device, a plurality of native food item data records that are associated with the geographic location of the dietary client device in the system database, in which each native food item data record of the plurality of native food item data records includes nutrient content per serving data; receiving, via the dietary client device, user input having data describing one or more selected native food item data records of the plurality of native food item data records and a number of servings of each of the one or more selected native food item data records; generating a native dish data record in the system database having data of the one or more selected native food item data records and the number of servings of each of the one or more selected native food item data records; determining nutritional characteristics data of the native dish data record; and displaying, via the GUI on the display screen of the dietary client device, an output that describes how the nutritional characteristic data of the native dish data record compares with the data of the recommended nutritional objective data record of the target diet data record.

In further embodiments, the system may perform operations that include: displaying, via the GUI of the dietary client device, a diet-setup input form; and receiving, via the GUI of the dietary client device, user input that modifies a recommended nutritional objective of the target diet data record.

In further embodiments, the system may perform operations that include: receiving, via the vendor client device, user input having data describing a custom food item, in which the data describing the custom food item includes nutrient content per serving data of the custom food item; and generating a custom food item data record in the system database having the nutrient content per serving data of the custom food item.

In further embodiments, the system may perform operations that include: generating a first unique scan code; and associating the first unique scan code with the custom food item data record in the system database.

In further embodiments, the system may perform operations that include: scanning the first unique scan code with the dietary client device; retrieving data of the custom food item data record from the system database; and displaying, via the GUI on the display screen of the dietary client device, the data of the custom food item data record.

In further embodiments, the system may perform operations that include: receiving, via the vendor client device, user input having data describing one or more custom food items, in which the data describing one or more custom food items includes nutrient content per serving data of the one or more custom food items; generating a custom dish data record in the system database having the data describing one or more custom food items and a number of servings of each of the one or more custom food items; and determining nutritional characteristics data of the custom dish data record.

In further embodiments, the system may perform operations that include: generating a second unique scan code; and associating the second unique scan code with the custom dish data record in the system database.

In further embodiments, the system may perform operations that include: scanning the second unique scan code with the dietary client device; retrieving data of the custom dish data record from the system database; and displaying, via the GUI on the display screen of the dietary client device, the data of the custom dish data record.

In further embodiments, the system may perform operations that include: displaying, via the GUI on the display screen of the dietary client device, an output that describes how the nutritional characteristics data of the custom dish data record compares with recommended nutritional objectives data of the target diet data record.

### BRIEF DESCRIPTION OF THE DRAWINGS

FIG. 1 depicts an illustrative example of some of the components and computer implemented methods which may be found in a system for integrated context-based personal diet management according to various embodiments described herein.

FIG. 2 shows a block diagram showing some software programs and components which may be found in the system according to various embodiments of the invention described herein.

FIG. 3A depicts an example of the basic data structure of a food item in a food database according to various embodiments described herein.

FIG. 3B illustrates an example data hierarchy of native food item data records according to various embodiments described herein.

FIG. 3C illustrates an example data hierarchy of custom food item data records according to various embodiments described herein.

FIG. 4 illustrates a flowchart of an example algorithm of some client device operations according to various embodiments described herein.

FIG. 5A depicts an example of a Vertical main-view design of a graphical user interface with all drop-down buttons closed according to various embodiments described herein.

FIG. 5B depicts an example of a Vertical main-view design of a graphical user interface with a Dish-Setup drop-down button expanded to reveal its input form area according to various embodiments described herein.

FIG. 6A depicts an example of a Stepper main-view design of a graphical user interface in different positions showing Diet-Setup sub view according to various embodiments described herein.

FIG. 6B depicts an example of a Stepper main-view design of a graphical user interface in different positions showing Dish-Setup sub view according to various embodiments described herein.

FIG. 6C depicts an example of a Stepper main-view design of a graphical user interface in different positions showing Calculate sub view according to various embodiments described herein.

FIG. 7A depicts an example toolbar of a graphical user interface of a dietary client device according to various embodiments described herein.

FIG. 7B illustrates an example toolbar of a graphical user interface of a vendor client device according to various embodiments described herein.

FIG. 8 depicts a block diagram of a preferred mode of operation for vendor client device access in the system according to various embodiments described herein.

FIG. 9A illustrates a diet-setup input form of a graphical user interface according to various embodiments described herein.

FIG. 9B illustrates an expanded view of the diet-selection list box of a graphical user interface according to various embodiments described herein.

FIG. 9C illustrates an expanded view of the calorie-specification list box of a graphical user interface according to various embodiments described herein.

FIG. 10A depicts an example of a dialog box for adding a new modification to the diet in the diet-setup input form of a graphical user interface according to various embodiments described herein.

FIG. 10B depicts an example of a dialog box for editing a pre-existing modification and for deleting it in the diet in the diet-setup input form of a graphical user interface according to various embodiments described herein.

FIG. 10C depicts an example of an expanded view of modification-selection list box of the dialog boxes in the diet-setup input form of a graphical user interface according to various embodiments described herein.

FIG. 10D depicts an example of an expanded view of the list box 138 for specifying the desired quantity (mass) of the modification in the dialog boxes in the diet-setup input form of a graphical user interface according to various embodiments described herein.

FIG. 11A depicts an example of a Dish-Setup input form of a graphical user interface for native food inputs showing empty form without any food items defined according to various embodiments described herein.

FIG. 11B depicts an example of a Dish-Setup input form of a graphical user interface for native food inputs showing the form with some food items defined according to various embodiments described herein.

FIG. 12A depicts an example of some dialog boxes for managing staple food items in the Dish-setup form showing a Dialog box for adding a new staple food item to the dish of a graphical user interface according to various embodiments described herein.

FIG. 12B depicts an example of some dialog boxes for managing staple food items in the dish-setup form dialog box of a graphical user interface for editing a pre-existing staple food item and for deleting it according to various embodiments described herein.

FIG. 13A shows an example of expanded views of list boxes of the dialog boxes of FIG. 12 with a List box 159 for selecting the desired number of food servings of a graphical user interface according to various embodiments described herein.

FIG. 13B shows an example of a list box of a graphical user interface for selecting a food-item according to various embodiments described herein.

FIG. 13C shows an example of a list box of a graphical user interface for selecting a country of origin of food items.

FIG. 14 illustrates an example of a dialog box of a graphical user interface used for scanning in custom food data according to various embodiments described herein.

FIG. 15A illustrates several example input views of a graphical user interface which may be used for selecting custom food items after a successful data scan according to various embodiments described herein.

FIG. 15B illustrates an example Dish-Setup custom-menu input form for food inputs and custom-menu selection dialog box of a graphical user interface according to various embodiments described herein.

FIG. 16 illustrates an example of a dialog box for displaying the calculation report on a graphical user interface according to various embodiments described herein.

FIG. 17 illustrates an example of a dialog box of a graphical user interface for the Settings view according to various embodiments described herein.

FIG. 18 illustrates an example of a dialog box of a graphical user interface managing the user's dish library according to various embodiments described herein.

FIG. 19A illustrates an example of a whole form dialog box of a graphical user interface managing the user's calculation library according to various embodiments described herein.

FIG. 19B illustrates an example of an expanded view of the calculation-selection list box of a graphical user according to various embodiments described herein.

FIG. 20 illustrates a block diagram showing an exemplary database structure of some data records of a system database which may be used in the system according to various embodiments described herein.

FIG. 21 depicts a block diagram showing another exemplary database structure of some data records of a system database which may be used in the system according to various embodiments described herein.

FIG. 22 shows a block diagram showing a further exemplary database structure of some data records of a system database which may be used in the system according to various embodiments described herein.

FIG. 23 depicts a block diagram of an example of a computer-implemented method of generating a system output that describes how nutritional characteristic data of a native dish data record compares with data of a recommended nutritional objective data record of a target diet data record according to various embodiments described herein.

FIG. 24 illustrates a block diagram of an example of a computer-implemented method of providing custom food item data to a client device according to various embodiments described herein.

FIG. 25 shows a block diagram of an example of a computer-implemented method of generating a custom dish data record in a system database according to various embodiments described herein.

FIG. 26 shows a block diagram of an example of a computer-implemented method of generating a system output that describes how nutritional characteristic data of a custom dish data record compares with data of a recommended nutritional objective data record of a target diet data record according to various embodiments described herein.

FIG. 27 depicts a block diagram illustrating an example of a server which may be used by the system as described in various embodiments herein.

FIG. 28 illustrates a block diagram illustrating an example of a client device which may be used by the system as described in various embodiments herein.

### DETAILED DESCRIPTION AND BEST MODE OF IMPLEMENTATION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

### Definitions

As used herein, the terms “computer” and “computing device” refer to a machine, apparatus, or device that is capable of accepting and performing logic operations from software code. The term “application”, “software”, “software code”, “source code”, “script”, or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software, with servers and client devices comprising exemplary computing devices.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anoto digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The terms “computer readable medium” and “computer readable memory” as used herein refers to any medium that participates in providing instructions to a processor for execution and/or participates in storing data which may be used by a processor. A computer readable medium or memory may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e., a “wireless network”) which may include Wi-Fi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigbee network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e., information and data from a database may be recorded into a medium on a data store).

Discussions of user interface elements will make use of normal every-day parlance (in all their various tenses) such as “click,” “press,” “button,” “list box,” “dialog box,” “window” etc., that should be readily familiar and understood by persons well versed in the arts. All user interface elements presented will not necessarily be drawn to scale.

Whenever we refer to a dialog box, unless otherwise stated, we shall ordinarily mean this to be a so-called modal dialog box (as opposed to a non-modal one) which precludes interactions with other parts of the system while it is open. Furthermore, an “X” in the upper right-hand corner of a dialog box or input form, unless otherwise stated, shall normally be understood as a button that closes (discards) the dialog box when it is pressed, without changing any part of the system. Another type of dialog box we shall have frequent recourse to, is the so-called docked dialog box, which overlaps (is docked within) a smaller area of the screen and acts modally over it. However, the user still has access to other parts of the user interface outside the region overlapped by the dialog box.

We shall use the term “session” to refer to all user activities from the time the user gains full access to the system up until such a time that access ends. A user may gain access following a successful authentication process and may subsequently terminate the session through a log-out process. The narrative generally may be concerned with only what happens during a session. We shall distinguish between 2 types of system users—a generic user (referred simply as the “user”) and a vendor user (referred simply as the “vendor”) that can create custom-food sources that may be uploaded and stored in the server to be accessible to all users of the system using their respective client devices. A vendor has access to all system features that are available to the generic user plus additional features that facilitate the creation of food content in the server.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

A new computer-implemented system and methods for integrated context-based personal diet management are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

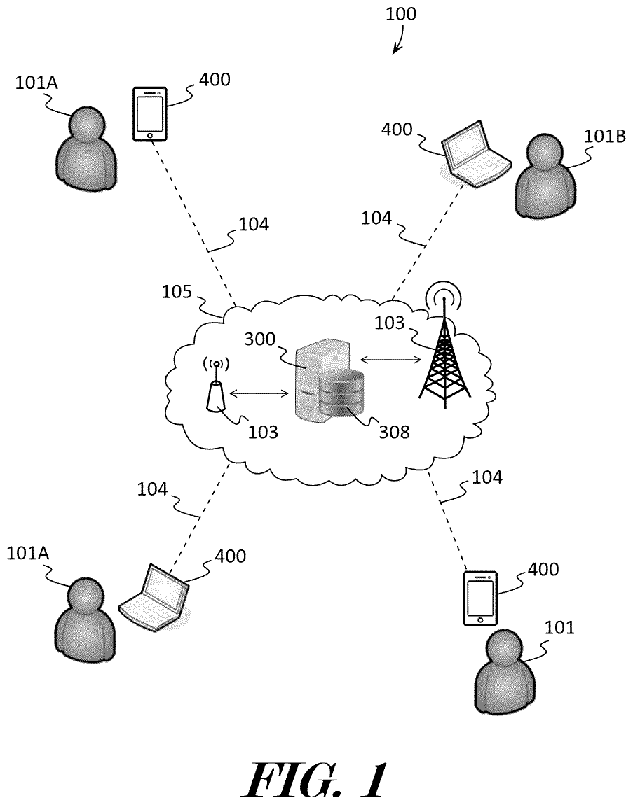

The present invention will now be described by example and through referencing the appended figures representing preferred and alternative embodiments. As perhaps best shown by FIG. 1, an illustrative example of some of the physical components which may comprise a computer-implemented system for integrated context-based personal diet management (“the system”) 100 according to some embodiments is presented. The system 100 is configured to facilitate the transfer of data and information between one or more users 101 via client devices 400 and servers 300, 300A, over a data network 105. Client devices 400 and servers 300, 300A, may send data to and receive data from the data network 105 through a network connection 104 with an access point 103. One or more data stores 308 accessible by a system server 300A, may contain one or more system databases 220. The data may comprise any information input into and generated by the system 100, including: information on or describing one or more users 101, such as user's name 101, email, phone number, picture, business information, client device identifying information etc.; information describing one or more food items; information describing one or more diets; information describing one or more dishes; information describing dietary calculations performed by the system 100; information describing recommended nutritional objectives generated by the system; and any other data and information described herein.

Client devices 400 and servers 300, 300A, may enable system 100 data exchange between users 101. In this example, the system 100 comprises at least one client device 400 (but preferably more than two client devices 400) configured to be operated by one or more users 101. Client devices 400 may include mobile devices, such as laptops, tablet computers, personal digital assistants, smart phones, and the like, that are equipped with a wireless network interface capable of sending data to one or more servers 300, 300A, via network communication with access to one or more data stores 308 over a network 105, such as a wireless local area network (WLAN). Additionally, client devices 400 may include fixed devices, such as desktops, workstations, and the like, that are equipped with a wireless or wired network interface capable of sending data to one or more servers 300, 300A, with access to one or more data stores 308 over a wireless or wired local area network 105. The present invention may be implemented on at least one computing device, such as a client device 400 and/or server 300, 300A, programmed to perform one or more of the steps described herein. In some embodiments, more than one client device 400 and/or server 300, 300A, may be used, with each being programmed to carry out one or more steps of a method or process described herein.

In some embodiments, the system 100 may be configured to facilitate the communication of information to and from one or more users 101 through their respective client devices 400, and system servers 300A. Users 101 of the system 100 may include dietary users 101A and vendor users 101B. A dietary user 101A may comprise an individual or entity that desires to manage their food intake and may use the system 100 to assist him or her in making educated food choices in real time. The client device 400 of a dietary user 101A may be referred to as a dietary client device 400A. A vendor user 101B may comprise an individual or entity that provides or sells food that may be consumed by dietary users 101A. For example, a vendor user 101B may comprise a restaurant or any other type of food vendor. The client device 400 of a vendor user 101B may be referred to as a vendor client device 400B.

In some embodiments, a user 101 of the system 100 interacts directly with a client device 400 to access available functions and data in the system 100. The system 100 may include one or more system servers 300A which may be a computer which resides in the cloud and is operated and maintained by a service provider (the System Operator or Sysop). Any standard cloud server with the appropriate hardware and software resources may function in this role, when properly configured and extended by persons well versed in the arts.

In preferred embodiments, the system 100 is configured for the planning and management of personal medical diet goals of dietary users 101A. In preferred embodiments, the system 100 provides the means for an individual dietary user 101A to monitor and analyze the nutritional value of his or her food intake through a graphical user interface of context-based inputs in a simple three-step process. The reliance on contexts leads to naturally-flowing user-experiences that mimic real life. The dietary user 101A of the system 100 has instant access to food nutritional data from a variety of sources, such as an “always on” geographical food source, as well as custom food sources that are available from food vendor users 101B, such as restaurants. The geographical source preferably represents the native food context (determined by client device location) for each dietary user 101A so that the system 100 presents a familiar food milieu to the user 101A—it is preferably available to all users 101. The custom sources may be opportunistic and allow a user 101 to gain access to food data by scanning a unique scan code 228, such as a food menu code provided by a vendor user 101B. In preferred embodiments, a vendor user 101B is a special user 101 of the system 100 that has access to additional interaction tools for creating custom food content (custom food item data records 223B and custom dish data records 225B). Thus, a dietary user 101A may use the system to assist him or her in making educated food choices in real time and food vendor users 101B may facilitate the extension of the system 100 data so that a user 101 has additional access to virtually an inexhaustible variety of custom-dish nutritional content.

In further embodiments, the system 100 may enable personal diet management for planning and managing of personal medical diet goals in a way that closely models real-life, and the system 100 may comprise a means by which users can:
(a) select a target diet data record 221A as a target diet from a plurality of medical diet data record 221 options that defines the proper context for determining the food nutritional goals to meet, and the manner of calculations and analysis to follow. Example diet options may be default (pre-provided) options (diabetic, heart-healthy, renal etc.), or custom diet definitions created by the user 101;
(b) assemble and manage a dish (dish data record 225) of food items drawn from a designated food context representing the geographical (native) food source of the dietary user 101A, preferably using the location of their dietary client device 400A as well as custom, opportunistic food sources read by the scanning of unique scan codes 228 (e.g., third-party food-vendor codes); and
(c) carry out a calculation that invokes a general-purpose solver that produces an output that describes how an assembled dish conforms with the nutritional recommendations of the target diet, and highlighting met and unmet diet goals, and in the latter case suggesting corrective dish modifications 227 and providing tools to assist the dietary user 101A make better dish choices.

In addition to individual use, the system 100 may be used as an effective aid for patient care by trained health-care professionals.

The system 100 preferably relies on the internet to function and utilizes a client-server system architecture. Users 101 interact with the system 100 through a client device. A system server 300A may provide a centralized service to multiple client devices 400. A system server 300A may be operated and maintained by a service provider. All aspects of this system 100 and its methods may be implemented in a plurality of forms optionally using standard software development tools and algorithms.

This system 100 may include client program 252 which may run on a client device 400 and which the user 101 can interact with to select a target diet that defines the primary context for calculations that follow and can assemble and manage a dish of food items drawn from a structured food context. The food context in the main menu of a graphical user interface 261 provided by include client program 252 associates the system 100 with a familiar geographical (or regional) food environment that constitutes its native food source. Furthermore, it incorporates scanned access to custom food offerings of third-party vendors. The native context uses a classification of food items into appropriate categories.

The system 100 preferably operates by a client-server framework within the global information-technology infrastructure popularly known as the internet, and parts of which are frequently (and loosely) referred to as the “cloud.” A typical system 100 scenario is the case of multiple client devices 400 communicating in real time with a single system server 300A (or a relatively small number of connected servers 300A working in concert).

In preferred embodiments, with the aim of making the system 100 more responsive: (a) diet calculations may be carried out locally in a client device 400; (b) user configuration data 223 and currently-needed food item data records 223 may be preloaded from the system server 300A to the client device 400, so that they can be processed faster; and (c) other intermittent bi-directional data exchange between the client device 400 and system server 300A occur only when needed.

In further embodiments of the system 100, a prospective food vendor user 101B may be associated with a special user (client) account having additional access to various (administrative) means for creating and transmitting to the server the following records:
(a) Nutritional information of the vendor's custom dishes (custom dish data records 225B) that may be derived from their recipes;
(b) food menus comprising the custom dishes offered by the vendor user 101B;
(c) unique scan codes 228 for facilitating access to vendor menus in the system database 220.

In the preferred embodiment, a vendor user 101B has access to all other functionalities that are normally available to a dietary user 101A, in addition to the above-listed ones.

The system database 220 contains all food-item data (food item data record 223) used by the system 100. These include native food data (native food item data record 223A) which may be defined by the primary geographical food context based on the location of each user 101, and custom food data (custom food item data record 223B) that may be accessed by users 101 by scanning unique scan codes 228 of food vendor users 101B, such as restaurants.

Client devices 400 may be configured to run three main functional components (modules): (1) Diet-Setup, (2) Dish-Setup, and (3) Calculate modules. The user 101 interacts with the client device 400 by navigating a convenient menu framework by means of a graphical user interface (GUI) 261 that presents various functional views and sub views as needed to the user 101 via the display screen 404A of their client device 400. The functional modules are independently accessible. However, a natural usage flow for them would be in the order listed above.

In the Diet-Setup module the user may choose a target diet (target diet data record 221A) of interest from a list of diet data records 221. The list items may include standard medically-prescribed diets such as Diabetic, Fat-Modified diets, among others, as well as custom user-defined diets. Associated with each diet data record 221 are recommended nutritional objectives data 222 for the diet. The user 101 may also specify a target calorie in this step and have the option of editing and modifying further the nutritional content of the chosen diet. The user 101 is able to create and manage a library of custom diets, that are always available for selection together with the default diet options.

In the Dish-Setup module the user 101 may create a dish (dish data record 225) of interest by entering food item data records 223 from the system database 220. These may be primary food entries, or custom entries associated with a scanned unique scan code 228 sent to the system server 300A by the client device 400. The user 101 is also able to create and manage a library of previously created dishes (dish library 232) and can import a dish data record 225 from this library 232 into the current session. In this way the user 101 can apply a history of dishes (such as a running collection of daily dishes) towards the current target diet (target diet data record 221A). Other possible embodiments of this step that may be utilized by the system 100, may include the maintenance of a “favorite list” of food item data records 223, and the recommendation of new food item data record 223 choices to the user 101 using machine-learning methods based on the user's food choice history.

By means of the Calculate module the user 101 initiates and executes a calculation by a client program 252, which produces useful output that conforms to the selected target diet data record 221A. This may be carried out by a general-purpose solver of the client program 252. The choice of diet provides the context for this step which generates an output that is appropriate to it. The solver invokes a calculation algorithm that is appropriate for the target diet data record 221A.

In preferred embodiments, the solver may apply calculation algorithms that may be derived from recommendations and guidelines provided by the United States Food and Drug Administration (FDA). The client program 252 analyzes the dish data record 225 specified by the user 101 against the recommended nutritional objectives data 222A of the target diet data record 221A. It tallies those dietary characteristics of the dish items and cross-references them against the requirements of the target. The calculation output may be in the form of a report that includes a comparative analysis of the tally results against target diet properties, with met and unmet dietary goals highlighted, and with corrective recommendations in the latter case, to bring it to within the constraints of the recommended nutritional objectives data 222A of the target diet data record 221A. Also included may be a detailed breakdown of the contributions of each dish component.

In further embodiments, the system 100 may be configured to generate calculation outputs that incorporate suggestions of alternative food servings to satisfy the constraints of the target diet. This includes accomplishing this task interactively, using input wizards or similar methods.

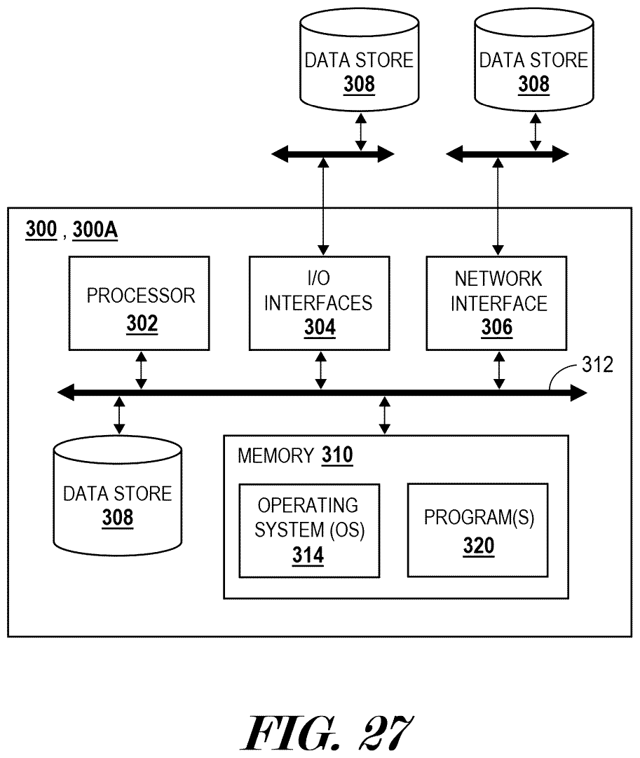

Referring now to FIG. 27, in an exemplary embodiment, a block diagram illustrates a server 300, 300A, of which one or more may be used in the system 100 or standalone and which may be a type of computing platform. A system server 300A is a server 300 that is configured to execute program instructions in order to perform one or more functions of the system 100. A server 300, 300A, may be a digital computer that, in terms of hardware architecture, generally includes a processor 302, input/output (I/O) interfaces 304, a network interface 306, a data store 308, and memory 310. It should be appreciated by those of ordinary skill in the art that FIG. 27 depicts the server 300, 300A, in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing program instructions. When the server 300, 300A, is in operation, the processor 302 is configured to execute software or programs 320 stored within the memory 310, to communicate data to and from the memory 310, and to generally control operations of the server 300 pursuant to the software or program instructions. The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 306 may be used to enable the server 300 to communicate on a network, such as the Internet, the data network 105, the enterprise, and the like, etc. The network interface 306 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 306 may include address, control, and/or data connections to enable appropriate communications on the network. A data store 308 may be used to store data.

The data store 308 is a type of memory and may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300, 300A, such as, for example, an internal hard drive connected to the local interface 312 in the server 300, 300A. Additionally, in another embodiment, the data store 308 may be located external to the server 300, 300A, such as, for example, an external hard drive connected to the I/O interfaces 304 (e.g., SCSI or USB connection). In a further embodiment, the data store 308 may be connected to the server 300, 300A, through a network, such as, for example, a network attached file server.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example Windows NT, Windows 2000, Windows XP, Windows Vista, Windows 7, Windows 8, Windows 10, Windows Server 2003/2008/2012/2016 (all available from Microsoft, Corp. of Redmond, WA), Solaris (available from Sun Microsystems, Inc. of Palo Alto, CA), LINUX (or another UNIX variant) (available from Red Hat of Raleigh, NC and various other vendors), Android and variants thereof (available from Google, Inc. of Mountain View, CA), Apple OS X and variants thereof (available from Apple, Inc. of Cupertino, CA), or the like.

The one or more programs 320 may comprise program instructions stored in one or more computer-readable memories and one or more processors 302 may be configured to execute the program instructions to cause the system 100 to perform operations described herein.

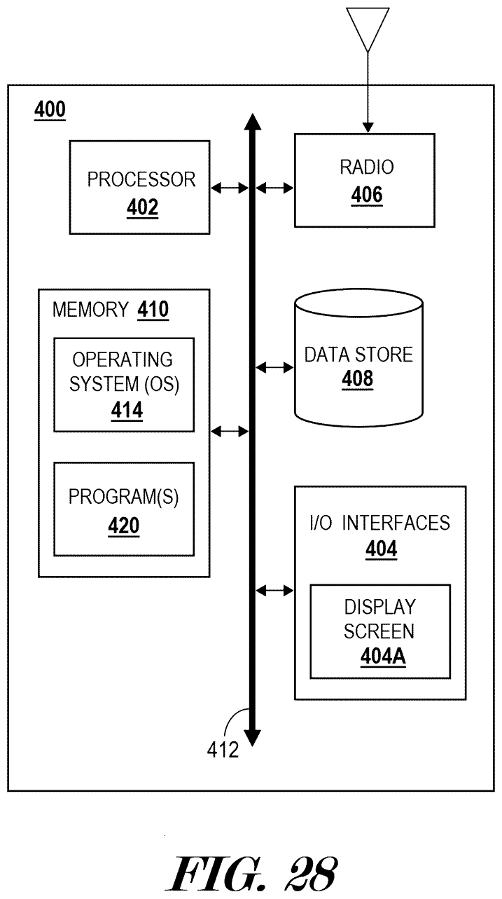

Referring to FIG. 28, in an exemplary embodiment, a block diagram illustrates a client device 400 of which one or more may be used in the system 100 or the like and which may be a type of computing platform. The client device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 28 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing program instructions. When the client device 400 is in operation, the processor 402 is configured to execute software or programs 420, having program instructions, stored within the memory 410, to communicate data to and from the memory 410, and to generally control operations of the client device 400 pursuant to the software or program instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, barcode scanner, voice recognition, eye gesture, and the like. System output can be provided via a display screen 404A, such as a liquid crystal display (LCD), light emitting diode (LED) display, touch screen display, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 28, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, 11, etc., iOS (available from Apple, Inc.), webOS (available from Hewlett Packard), Blackberry OS (Available from Research in Motion), and the like.

The one or more programs 420 may comprise program instructions stored in one or more computer-readable memories and one or more processors 402 may be configured to execute the program instructions to cause the system 100 to perform operations described herein. The programs 420 may be configured to provide end user functionality with the client device 400. For example, exemplary programs 420 may include, but not limited to, a web browser, social networking applications, streaming media applications, games, mapping and location applications, electronic mail applications, financial applications, and the like. In a typical example, the end user typically uses one or more of the programs 420 along with a network 105 to exchange information with the system 100.

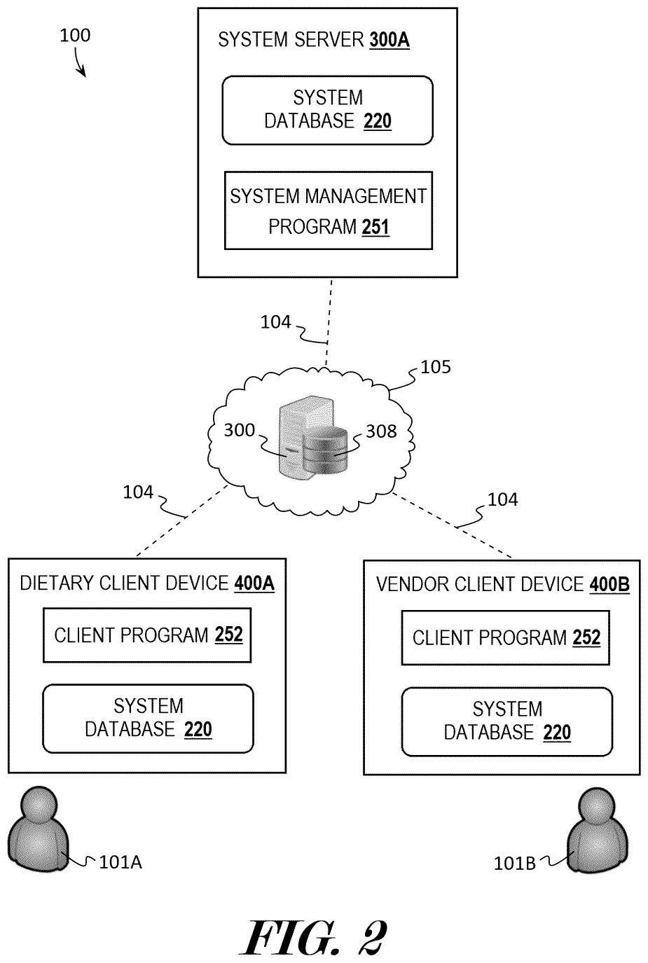

Referring now to FIG. 2 a block diagram showing some software programs 251, 252, and components which may be found in a system 100 and which may optionally be configured to run on one or more system servers 300A and/or client devices 400 according to various embodiments described herein are illustrated. A system server 300A and client devices 400 preferably may be in wireless electronic communication through a network 105. The programs 251, 252, may be in electronic communication so that data may be readily exchanged between the programs 251, 252.

In this and some embodiments, one or more servers 300 may be configured to run a system management program 251 while client devices 400 may be configured to run a client program 252. A system management program 251 and/or client program 252 may be configured to run on one or more system servers 300A, such as by being cloud based, and/or client devices 400, such as by being local to client device(s) 400 of a user 101, according to various embodiments described herein. It should be understood that the functions attributed to the programs 251, 252, described herein are exemplary in nature, and that in alternative embodiments, any function attributed to any program 251, 252, may be performed by one or more other programs 251, 252, or any other suitable processor logic.

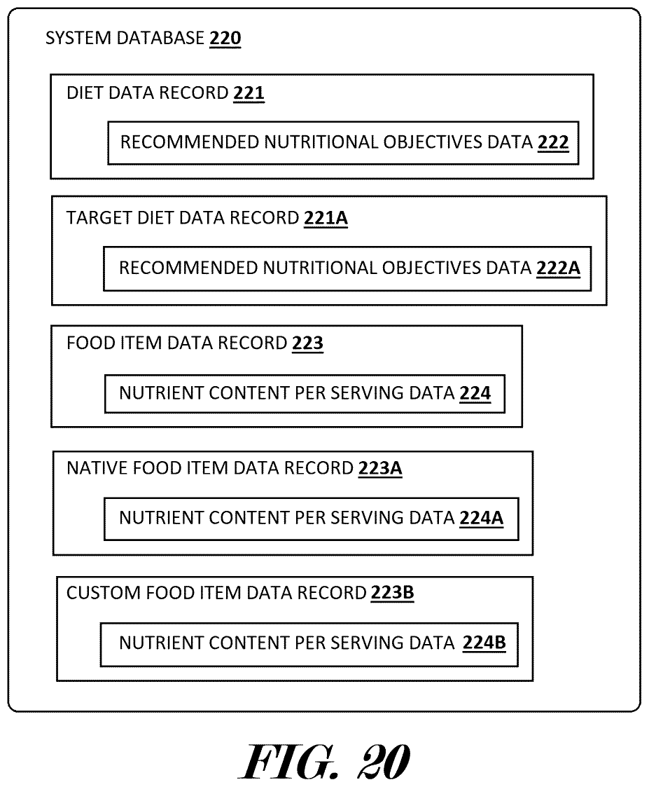

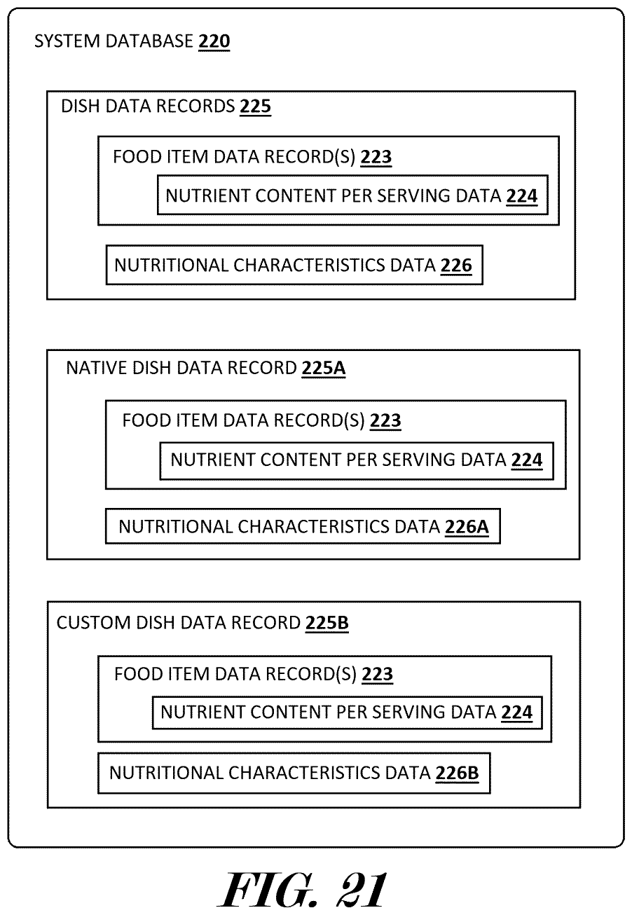

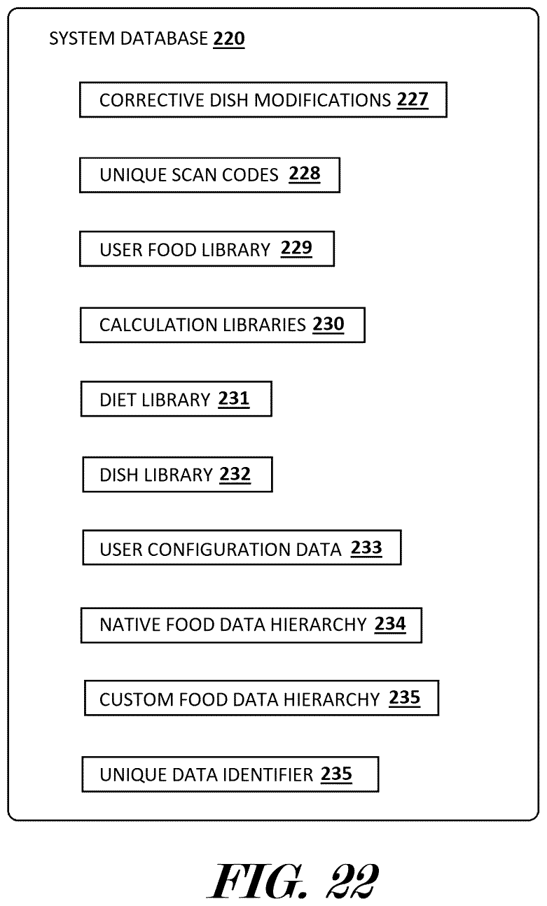

Referring to FIGS. 2 and 20-22, the system 100 may comprise one or more databases, such as a system database 220, which may be stored on a data store 308 accessible to one or more programs 251, 252. In some embodiments, a system database 220 may comprise any information input into and generated by the system 100, including: information on or describing one or more users 101, such as user's name 101, email, phone number, picture, business information, client device identifying information etc.; information describing one or food items; information describing one or more diets; information describing one or more dishes; information describing dietary calculations performed by the system 100; information describing recommended nutritional objectives generated by the system; and any other data and information described herein.

In preferred embodiments, the system database 220 may comprise a plurality of diet data records 221. Each diet data record 221 may contain data describing a diet that the system 100 may offer to a dietary user 101A, including standard medically-prescribed diets such as Diabetic, Fat-Modified diets, among others, as well as custom user-defined diets. This data may include recommended nutritional objectives data 222 which be data describing one or more recommended nutritional objectives (nutritional goals) of the diet. As an example, a diabetic diet data record 221 may have recommended nutritional objectives data 222 comprising Carbohydrates should constitute 50%-60% of total calories and Proteins should constitute 15%-20% dietary intake. A dietary user 101A may select a diet data record 221 as their target diet data record 221A, and the recommended nutritional objectives data 222A of the target diet data record 221A may be used for system calculations and system generated output which may aid the dietary user 101A in practicing the diet.

In preferred embodiments, the system database 220 may comprise a plurality of food item data records 223, and each food item data record 223 may comprise data describing the food item of the food item data record 223, such as nutrient content per serving data 224. Generally, nutrient content per serving data 224 may be data describing the nutrients and national data in a serving of a food item. The nutrient content per serving data 224 may also have data describing a serving size of the food item. Preferably, food item data records 223 may be associated with location data 237 so that each food item data record 223 may be associated with a geographic region, country, location etc.

Food item data records 223 may include native food item data records 223A and custom food item data records 223B. Native food item data records 223A may comprise data on food items which may be defined by the primary geographical food context of the food. Custom food item data records 223B comprise data on food items which may be added to the system 100 by vendor users 101B and which may describe food items made or sold by the vendor users 101B. Custom food item data records 223B may each be associated with a unique data identifier 236. Preferably, native 223A and custom 223B food item data records may be associated with location data 237 so that each native 223A and custom 223B food item data record may be associated with a geographic region, country, location etc. Native 223A and custom 223B food item data records may each comprise nutrient content per serving data 224A, 224B, describing the food item of the native 223A or custom 223B food item data record.

In preferred embodiments, the system database 220 may comprise a plurality of dish data records 225, and each dish data record 225 may have data describing a dish having one or more servings of food items. Each dish data record 225 may comprise or be associated with one or more food item data records 223 and the nutrient content per serving data 224 of the one or more food item data records 223. Each dish data record 225 may also comprise or be associated with nutritional characteristics data 226 which may describe the number of servings of each food item in the dish and the total of all the nutrient content per serving data 224 of the one or more food items in the dish. Native dish data records 225A may comprise data of at least one native food item data record 223A, while custom dish data records 225B may comprise data of one or more custom food item data records 223B input by a vendor user 101B.

In preferred embodiments, the system database 220 may comprise a plurality of corrective dish modifications 227. Corrective dish modifications 227 may comprise data describing changes that a user 1101 may make to bring a dish of a dish data record 225 within the constraints of a target diet.

In preferred embodiments, the system database 220 may comprise a plurality of unique scan codes 228. Preferably, each custom food item data record 223B and each custom dish data record 225B may be associated with a unique scan code 228. A unique scan code 228 may comprise any type of machine readable code, such as a food-vendor barcode, QR code, etc. Preferably, each unique scan code 228 may be associated with a generated unique data identifier 236 that may encode universal resource locator (URL) information that can be retrieved by scan code readers of dietary client devices 400A to facilitate access of dietary user 101A to custom food data of the system 100.

In preferred embodiments, the system database 220 may comprise a plurality user food libraries 229. Preferably, the system database 220 may comprise a user food library 229 for each dietary user 101A that may include data such as previously saved and entered food input.

In preferred embodiments, the system database 220 may comprise a plurality of calculation libraries 230. Preferably, the system database 220 may comprise a calculation library 230 for each dietary user 101A that may include data such as determined nutritional characteristics data of the dish data records 225.

In preferred embodiments, the system database 220 may comprise a plurality of diet libraries 231 and dish libraries 232. Preferably, the system database 220 may comprise a diet library 231 and a dish library 232 for each dietary user 101A. The diet library 231 may include data describing previously saved custom diet definitions of one or more diet data record 221 by the user 101 and in which the user 101 may add new diet data records 221 or delete old ones, and the dish library 232 of which contents comprises of previously saved dish data records 225 of interest by the user 101 and in which the user 101 may add new dish data records 225 or delete old ones, and the calculations library 230 of which contents comprises of previously saved diet, dish and calculation-report records and in which the user 101 may add new records and delete old ones.

In preferred embodiments, the system database 220 may comprise a plurality of user configuration data 233 records. Preferably, the system database 220 may comprise user configuration data 233 for each user 101 which may describe settings and preferences, such as graphical user interface 261 preferences, selected by each user 101. This may be stored in a database and/or in an externally-attached storage. Configuration data optionally includes system access data (for embodiments that require user authentication), user diet 231, food 229, and calculation library 230 data.

In preferred embodiments, the system database 220 may comprise a native food data hierarchy 234 for native food item data records 223A having data describing or following a top-bottom relationship of country, food-category and food object and with entries at each level of the hierarchy 234 having unique identifiers associated with them.

In preferred embodiments, the system database 220 may comprise a custom food data hierarchy 235 for custom food item data records 223A having data describing or following a top-bottom relationship of vendor user 101B, menu and food object and with entries at each level of the hierarchy 235 having unique identifiers associated with them.

In preferred embodiments, the system database 220 may comprise a plurality of unique data identifiers 236, and each unique data identifier 236 may be associated with food item data record 223 so that the unique data identifier 236 may be unique to the food item of the food item data record 223.

In preferred embodiments, the system database 220 may comprise a plurality of location data 237. Preferably, food item data records 223 may be associated with location data 237 so that each food item data record 223 may be associated with a geographic region, country, location etc.

In preferred embodiments, the system 100 may include a system management program 151 which may comprise program instructions stored in one or more computer-readable memories 310, 410, and executable by one or more processors 302, 402. Preferably, a system management program 151 may be executable by a processor 302 of a system server 300A. A system management program 151 may be configured to add, delete, and modify data in the system database 220. A system management program 151 may send data to and receive data from the client programs 152, and may enable data to be exchanged between client programs 152. A system management program 151 may be configured to determine nutritional characteristics data of dish data records 225. A system management program 151 may be configured to generate an output that describes how nutritional characteristic data 226 of a dish data record 225 compares with data of a recommended nutritional objective data record 222A of a target diet data record 221A. A system management program 151 may be configured to generate data describing how at least one nutritional characteristic of a dish data record 225 does not meet at least one recommended nutritional objective 222A of a target diet data record 221A. A system management program 151 may also be configured to perform other functions as described herein.

In preferred embodiments, the system 100 may include a client program 152 which may comprise program instructions stored in one or more computer-readable memories 310, 410, and executable by one or more processors 302, 402. Preferably, a client program 152 may be executable by a processor 402 of a client device 400. Generally, a client program 152 may be configured to operate an I/O interface 404, such as a display screen 404A (optionally of a touchscreen interface), of a client device 400 operated by a user 101 in order to provide information to and receive information from the user 101. A client program 152 may be configured to provide geolocation data of a user's 101 client device 400 and other location data described herein. A client program 152 may be configured to generate a graphical user interface 261 on the display screen 404A of a client device 400 which may be used by a user 101 to provide user input and to display system output via the client device 400. A client program 152 may also be configured to perform other functions as described herein.

In preferred embodiments, a client program 152 may be configured to generate a graphical user interface (GUI) 261 on the display screen 404A of a client device 400. The GUI 261 may be a system of graphical menus and views by which the user 101 interacts with the client device 400. In preferred embodiments, a GUI 261 may be configured to provide several views and controls including:
(a) diet-setup input form 123 and related views by which a user 101 is able to select a target diet 221A and make modifications to its set of recommended nutritional objectives 222A e.g., calorie value and its other nutritional properties;
(b) dish-setup form 144 and related view by which the user 101 can create a dish data record 225 of interest composed of several food items 223, such as native food items and custom food items and/or by importing and merging previously saved food input from a user food library 229, and by which the user 101 can otherwise modify and delete the said food items 223 as needed;
(c) a calculate button 122 and related views by which the user 101 can invoke a solver of a client program 252 appropriate to the target diet 221A, view an appropriate report generated by the solver, and is able to export desired diet 221, dish 225, and calculation records to a user library of such data;
(d) a scan button 126 and related views by which a user 101 is able to read unique scan codes 228 e.g., food-vendor barcode, QR code, or other similar code, in order to gain access to custom food menu data (nutritional characteristics data 226B) provided by a vendor user 101B and stored at a system server 300A, and to use such as a source of food items 223 for creating dish data records 225;
(e) a settings button 125 and related views by means of which the user 101 can manage diet 231, dish 232, and calculation libraries 230, and the diet library 231 of which contents comprises of previously saved custom diet definitions of one or more diet data record 221 by the user 101 and in which the user 101 may add new diet data records 221 or delete old ones, and the dish library 232 of which contents comprises of previously saved dish data records 225 of interest by the user 101 and in which the user 101 may add new dish data records 225 or delete old ones, and the calculations library 230 of which contents comprises of previously saved diet, dish and calculation-report records and in which the user 101 may add new records and delete old ones; and
(f) (when applicable) a vendor-access button 210 and related views by means of which a vendor user 101B is able to create and upload to a system server 300A custom food data (custom food item data record 223A) and menu data (custom dish data record 225B) and unique scan codes 228 for accessing this data.

In preferred embodiments, the system 100 incorporates additional logic features to ensure a responsive user experience. For example, user 101 configuration data may be pre-loaded from a system server 300A and stored in the client device 400 at the start of a user 101 session. Blocks of food data may also be downloaded as needed from a system server 300A to be processed locally by the client device 400. Only relatively small data transfers may then subsequently occur between client device 400 and a system server 300A as needed. Calculation of diets may also be advantageously carried out locally in the client device 400, as for example when the user 101 modifies and saves a user configuration data 233. Data transfer from the client device 400 to the system server 300A may be arbitrated by dedicated functions within the code libraries. The exchanged data should include suitable control data, so that appropriate service requests may be efficiently fulfilled at the client device 400 and system server 300A by a system management program 251 and client program 252.

Food data that is accessible to all system users 101, is stored in the system database 220. In preferred embodiments, the system database 220 is implemented as the storage component of a relational database system (RDS) that the system management program 251 and client programs 252 can access. A RDS uses special query language constructs—such as structured query language (SQL) queries—to efficiently retrieve and store data. In further embodiments, the system database 220 may comprise any type of database.

In preferred embodiments, two kinds of food item data record 223 and dish data records 225—native and custom—may be stored in the system database 220. The native set, native food item data records 223A and native dish data records 225A, may be generic data that represents the primary geographical food context of the client device 400 of the user 101, preferably determined by the location of the client device 400. The custom set, custom food item data records 223A and custom dish data records 225B, may be food data provided by food vendors users 101B, such as restaurants, that is accessible to patrons of such vendors. This access will preferably take place after a patron (dietary user 101A) scans a unique scan code 228 (such as a barcode or a QR code on a menu), using their dietary client device 400A.

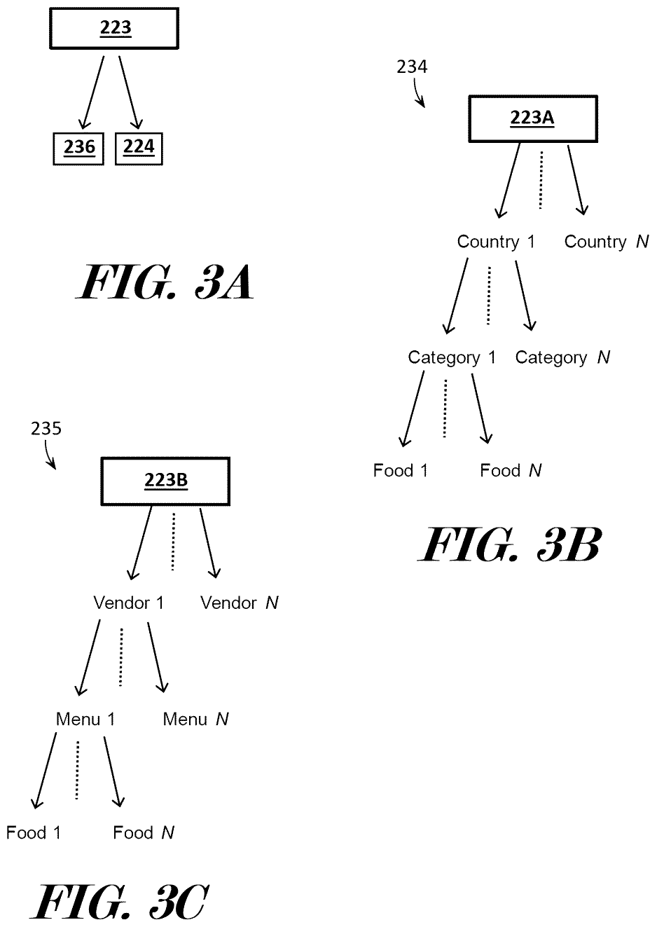

In preferred embodiments and as shown in FIGS. 3A-3C, the system database 220 may be stored on a system server 300A and is preferably includes: (a) a plurality of food item data records 223 which may comprise a basic food object definition for storing individual food entries that is characterized by a unique food identifier (ID) and nutrient content per serving data 224 for each food item data records 223; (b) a native food data hierarchy 234 for native food item data records 223A following a top-bottom relationship of country, food-category and food object and with entries at each level of the hierarchy 234 having unique identifiers associated with them; and (c) a custom food data hierarchy 235 for custom food item data records 223A following a top-bottom relationship of vendor user 101B, menu and food object and with entries at each level of the hierarchy 235 having unique identifiers associated with them.

An example of the data hierarchy of native and custom data sets is illustrated in FIGS. 3A-3C. Each node of the hierarchies 234, 235, has a unique data identifier 236 associated with it. Basic food data may be available in a consistent format internally in each client device 400 regardless of its source and may be characterized in the system database 220 by the unique data identifier 236 and its nutrient content per serving data 224 (FIG. 3A). The nutrient content per serving data 224 may include the amount of food nutrients (Calorie, Protein, Fat, Cholesterol Carbohydrate, Sodium, Potassium, Iron, etc.) per serving of the food item.

At the highest level of the native data, food items of native food item data records 223A may be identified by country of origin (FIG. 3B). For example, an implementation of a client device 400 may cover a geographical region consisting of individual countries or larger groupings of countries such as the whole world, continents, continental subregions etc. Native food entries may be further classified into familiar food categories for the region they represent. For example, food may be categorized as “entrees,” “staples,” “accompaniments,” “desserts,” “fruits and vegetables,” or other appropriate categories.

A unique food-vendor access code may be the highest level of classification in the custom data set (FIG. 3C). This references the food-menu data associated with the food vendor users 101B.

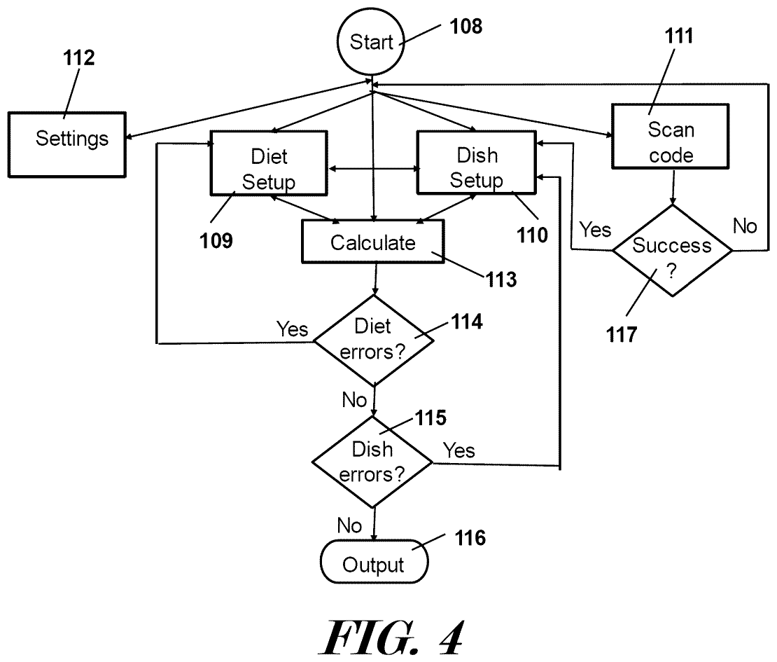

A flowchart illustrating example operations of a client device 400 is shown in FIG. 4. From the Start state 108, a user 101 can independently access different data input views (via steps 109. 110, 111, 112, etc.) that may be generated on the graphical user interface 261 of the user's 101 client device 400 via a client program 252: A Diet-Setup step 109, to specify a target diet data record 221A as a target diet, a Dish-Setup step 110, to manually specify a dish, a Scan-code step 111, to initiate the scanning in of custom food data via scanning of a unique data identifier 236 via the user's 101 client device 400, a Settings step 112, to manage user configuration data 233, or a Calculate step 113 to initiate a diet calculation. A Calculation is typically started by pressing a button, which herein (without loss of generality) is assumed to be the case for the purposes of our further discussion.

At the scan-code step 111, a user 101 can specify a custom-sourced dish by scanning a unique data identifier 236 provided by a vendor user 101B, such as on a menu. The received scan input preferably undergoes two verification steps at the decision step 117: (1) a compatibility test to confirm that it is well formatted as required by the system 100, and (2) a check of the availability of all necessary food-vendor data at a system server 300A that are associated with the received input. Following a successful scan, food data is suitably displayed to the user 101 via the graphical user interface 261 for further manual refinement at step 110.

Pressing the Calculate button 205 in step 113 invokes a calculator program of a client program 252 that first verifies the accuracy of the diet inputs (at decision step 114) and the dish inputs (at decision step 115). It should be noted in the passing that the order in which these checks are carried is of no consequence. If there is an error in the diet input, the calculation is halted, and the user 101 is returned to the diet-specification step 109 via the graphical user interface 261. This will be the case if an incorrect target diet has been selected, and/or if incorrect diet-related input values were specified. Similarly, the calculation is halted, and the user 101 is returned to the dish-input step 110 if no dish has been specified.

The calculation proceeds to completion if no errors have been detected, culminating in the transition to the Output step 116.

An alternative preferred embodiment of the system 100 that is suitable for (but not limited to) mobile apps, would dispense with an explicit Calculate step 113 altogether, and instead have it integrated with Output step 116. The latter will then be continuously updated on-the-fly with new output generated for changing, non-erroneous diet and dish inputs.

The Settings step 112 enables the user to manage configuration data such as custom (user-defined) diet library data 231, dish-library data 232, calculation-library data 233, and other session-control data.

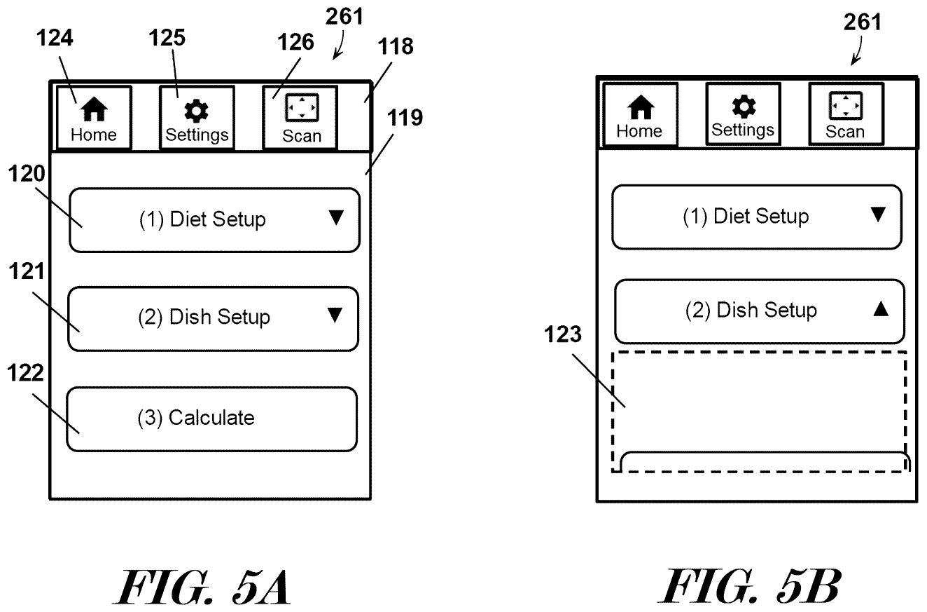

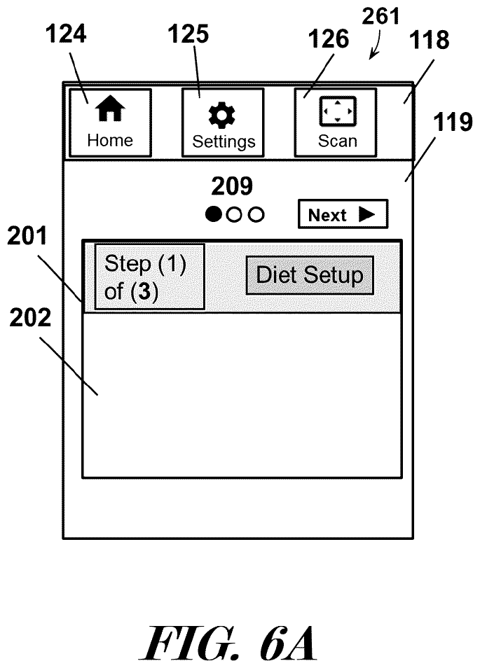

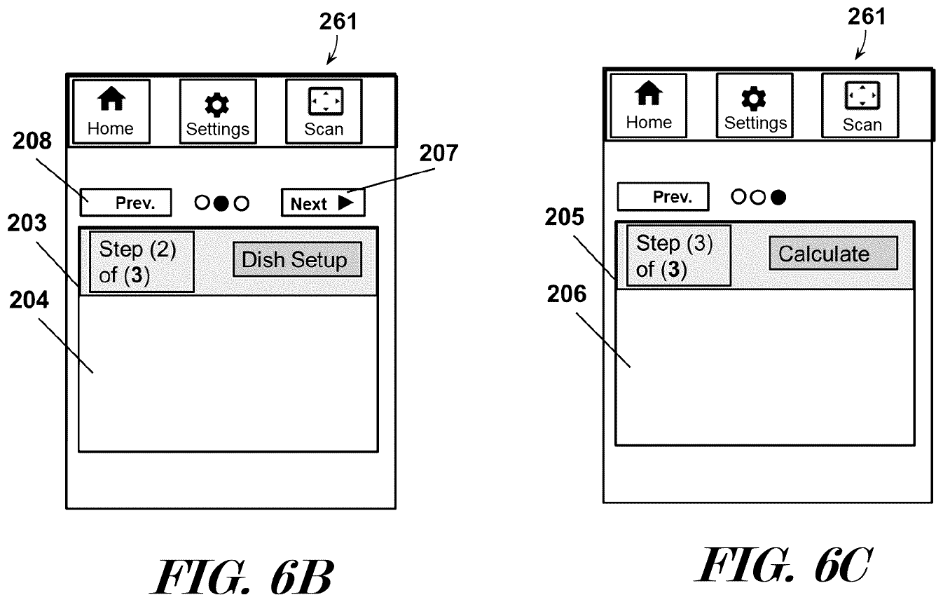

FIGS. 5 and 6 depict example design layouts of the main application view of the graphical user interface 261 of the system 100, in embodiments that are preferably suitable for mobile apps on mobile client devices 400. These may be the initial views at the start of new app sessions. Henceforth we shall refer to these main-view graphical user interface 261 embodiments as Vertical (FIGS. 5A and 5B) and Stepper (FIG. 6) designs respectively.

Vertical offers 3 “drop-down” buttons for accessing the data input areas of the view. Stepper employs a wizard-like stepping method for accessing the data input areas. The main view of both Vertical and Stepper preferably includes two functional areas: A toolbar 118 and a main user input pane (or simply, the user pane) 119.

Other embodiments, employing variations in the type, placement, iconography and naming conventions of the user-interface elements of graphical user interface 261, in this and other views, and for all possible implementation platforms, are contemplated by this invention.

FIGS. 5A and 5B depict an example of a Vertical main-view design of the graphical user interface 261, with FIG. 5A showing a view with all drop-down buttons closed and FIG. 5B showing a view of the Dish-Setup drop-down button expanded to reveal its input form area 123.

FIGS. 6A-6C depict examples of a Stepper main-view design of the graphical user interface 261 in different positions with FIG. 6A showing the Diet-Setup sub view; FIG. 6B showing the Dish-Setup sub view; and FIG. 6C showing the Calculate sub view.

We first give a brief exposition of the Vertical main view (FIGS. 5A and 5B) and then highlight its design differences with the Stepper (FIGS. 6A-6C) approach. The user pane 119 of the Vertical view preferably, contains the following three buttons: (1) The Diet-Setup button 120, that displays an input form (view) for specifying a target diet; (2) the Dish-Setup button 121, that displays an input area made up of controls for specifying (building) a dish of interest by making selections from a food library; (3) the Calculate button 122, that initiates a calculation process, which culminates (in the absence of any diet or dish-specification errors) in the generation and display of a report appropriate for the specified target diet.

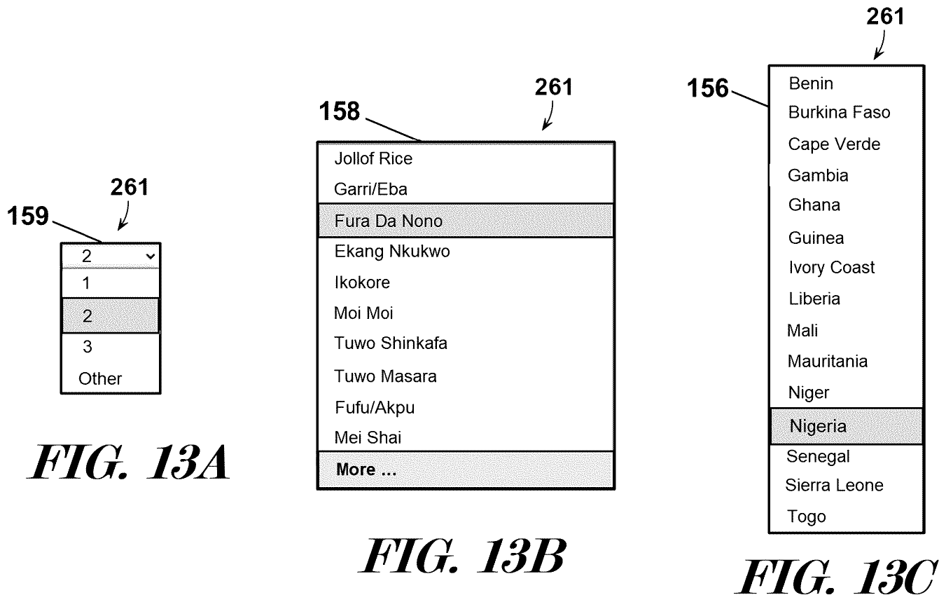

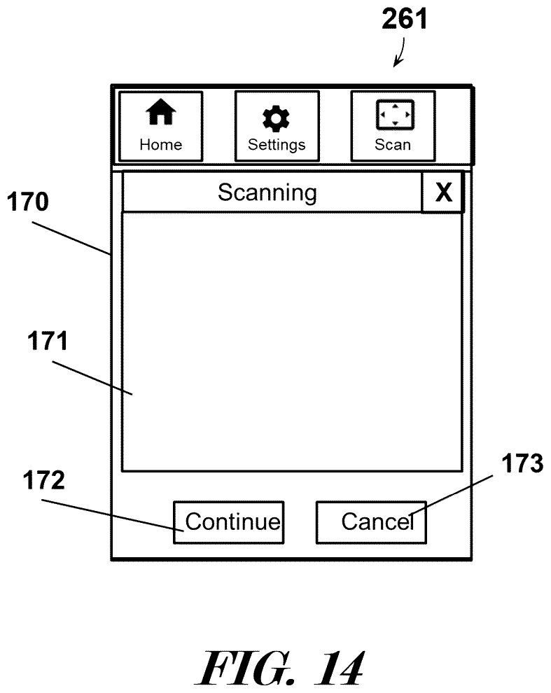

Buttons 120 and 121 may preferably be “drop-down” buttons. Pressing them shows (expands) or hides (as the case may be) their associated input areas located below them, as illustrated in FIG. 5B for the Dish-Setup button 121, with expanded diet-setup input form area 123. The report produced by pressing the Calculate button 122, preferably is presented in a view which overlaps the user pane (see FIG. 13 below). The onset of the calculation process may be aborted if any diet input is in error, or if the dish input is empty. In this case the calculation is terminated, and the diet or dish input areas are expanded with an appropriate error message displayed.

FIGS. 6A-6C depict 3 different sub views of the Stepper main-view design. The sub views follow in the sequence: (1) Diet-Setup 201 with data input area 202, (2) Dish-Setup 203 with data input area 204 and (3) Calculate 205 with action area 206. This sequence can be stepped through in order using the Next 207 and Previous 208 buttons. Clicking the Next button navigates to the step immediately following the current one in the sequence, while clicking the Previous button navigates back to the previous step.

Preferably, the data input of the current sub view is checked for errors whenever the Next button 207 is clicked. If any of the input data is erroneous, the navigation is preferably then aborted, and the user alerted of this so that the data may be corrected. The last (Calculate) step can automatically generate the necessary output results and also provide a means for the user to manually do so. Preferably, also available, are option (radio) buttons 209 that permit the navigation of the sub views in a random manner without error checking.

In some embodiments, the toolbar 118, may preferably contain several buttons for navigating to different views of the app. Pressing the Home button 124 redisplays the main view (FIG. 5A). Pressing the Settings button 125, or the Scan button 126 displays views for managing user configuration data 233, or scanning custom food data, in views overlapping the user pane. The graphical user interface 261 of the system 100 may include the optional inclusion in the toolbar 118 or in other convenient areas of the invention, additional buttons and other elements playing other roles, such as a “Help” button, a “Logout” button (if needed) etc.

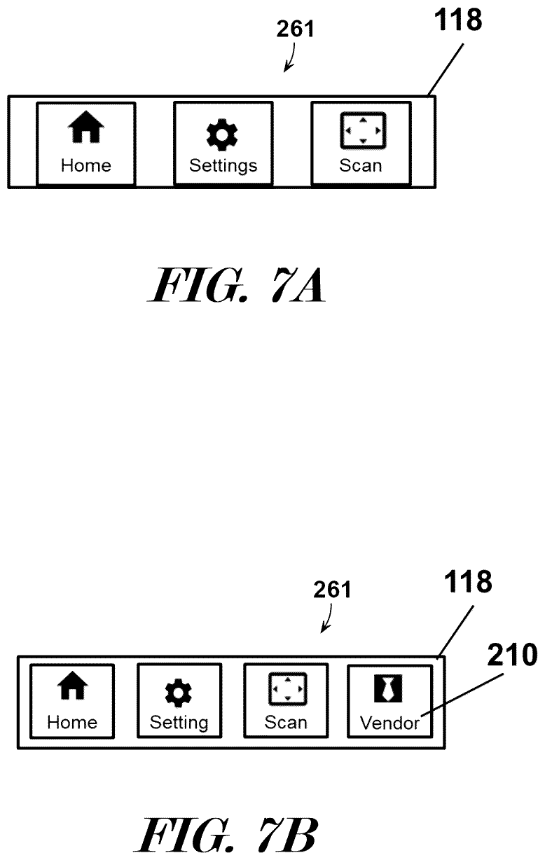

In preferred embodiments, a logged-in vendor user 101B may be presented with an extra vendor-access button 210 in the toolbar 118 as illustrated in FIGS. 7A and 7B. This vendor-access button 210 (FIG. 7B) is not available to dietary users 101A (FIG. 7A). Clicking vendor-access button 210 navigates to a vendor-access view (not shown).

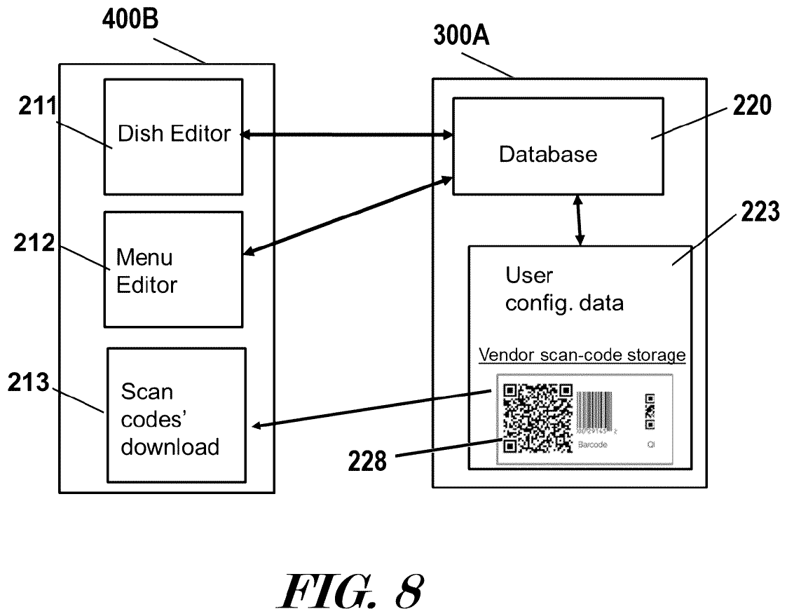

The block diagram of FIG. 8 illustrates a preferred mode of operation for vendor user 101B access in the system 100. In preferred embodiments, the vendor access occurs at the vendor view displayed by clicking the Vendor button 210 (FIG. 7B).

The vendor user 101B preferably uses an appropriate dish-editor 211 and menu-editor tool 212 tools of the graphical user interface 261 to create and exchange custom food data of custom food item data records 223A and custom dish data records 225B with a system server 300A in a system-compatible format. Data received by the system server 300A is preferably stored in the system server's 300A system database 220. Preferably, as part of this data transfer phase, specialized processes of the system management program 251 will be spurned to create associated unique data identifiers 236 or data-access scan codes (QR codes, barcodes etc.) that are preferably saved in the user data configuration storage (233. The store dish and menu data in storage in the system database 220 may preferably be retrieved, updated and otherwise modified by the vendor user 101B via their vendor client device 400B as needed.

The generated unique data identifiers 236 preferably encode universal resource locator (URL) information that can be retrieved by scan code readers of dietary client devices 400A to facilitate access of dietary user 101A to custom food data of the system 100. A vendor user 101B will preferably have access to the unique data identifiers 236 and have a means, e.g., button 213, to be able to download and publish them as needed for the benefit of its patrons (dietary users 101A). The Scan view functionality is discussed in greater detail below.

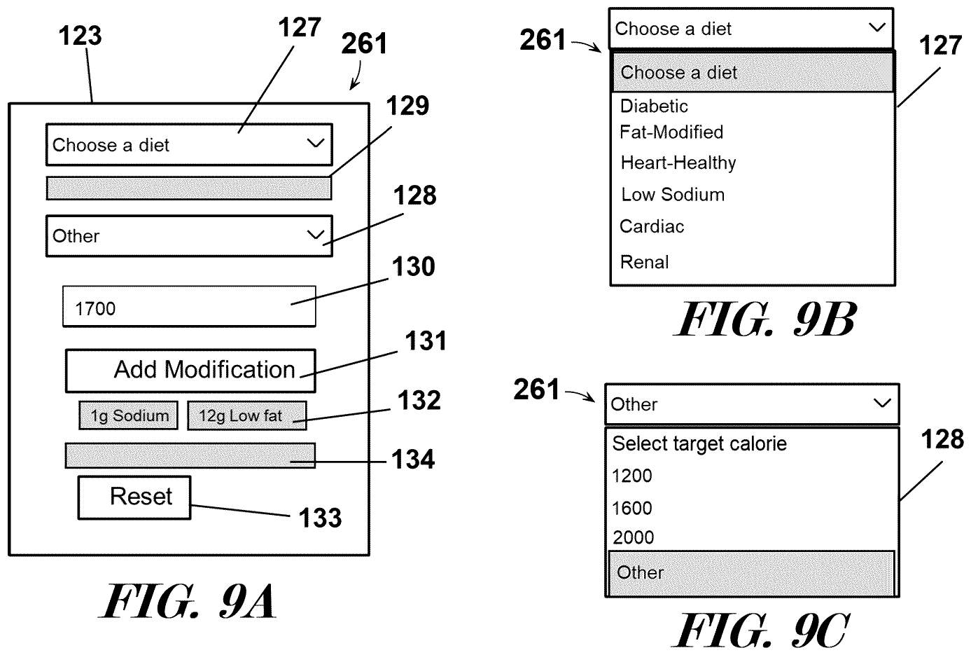

An example of the user-interface elements may be generated on a graphical user interface 261 of the diet-setup input form 123 is shown in FIGS. 9A-9C, in which FIG. 9A depicts the whole form. This diet-setup input form 123 is the input area associated with an open (expanded) diet-setup drop-down button 120 of the user pane 119 of the Vertical main-view layout (FIGS. 5A and 5B) as well as of the input area 202 of the Stepper layout (FIG. 6A). The target diet and the target calorie value are selected from the list boxes 127 and 128, shown in their expanded views in FIG. 6B and FIG. 6C. Each entry of list 127 represents a standard medically-defined diet of a diet data record 221, or a user-defined custom diet of a diet data record 221. In preferred embodiments, a summary description of a selected diet is displayed in the summary description area 129 below list box 127 in the diet-setup input form 123.

Additional preferred embodiments may organize the standard and custom lists separately from each other, and a user 101 may preferably then initiate and set the selection from a diet list of interest using a toggle switch mechanism.

Pre-defined calorie values may be selected from list 128. By default, a target calorie of 2000 cal. may be pre-selected. This is the value recommended by the FDA for general nutritional advice of a healthy adult. A value (other than the predefined ones) may also be manually entered by selecting “Other” from list 128. This displays the input box 130 (which will otherwise be hidden for other selections), that allows the user 101 to enter a different calorie value.

Additional modifications may be made to the diet by pressing the Add-Modification button 131. This opens a dialog box for accomplishing this. Added modifications are listed in the area 132 of the diet-setup input form 123, below the Add Modifications button 131. Clicking an item in area 132, opens a dialog box for editing or deleting the item. The summary-description area 129 is updated accordingly to reflect any new modifications to the diet. Clicking the Reset button 133, returns all diet inputs of the diet data record 221 to their default values.

FIGS. 9A-9C depicts an example of a Diet-Setup input form 123 with FIG. 9A showing the whole form; FIG. 9B showing the expanded view of the diet-selection list box 127; and FIG. 9C showing expanded view of the calorie-specification list box 128.

In further preferred embodiments, the graphical user interface 261 may include the provision of a Save button (not shown in FIGS. 9A-9C) that displays a dialog box (not shown) that enables the user 101 to save the current dish to the user's diet library 231 and/or dish library 232, which preferably is a repository of custom diet definitions. The dialog box includes a provision for entering an identifying name input for the diet definition being saved.

Any diet error messages (that may arise during a calculation) may preferably be displayed in the area 134 above the Reset button 133.

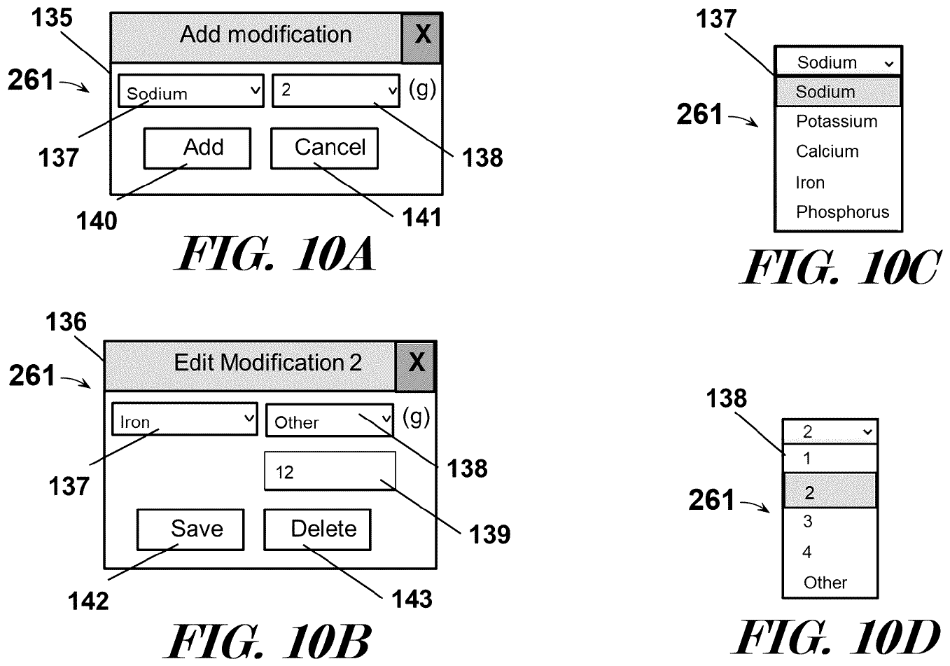

Two simple examples of dialog boxes for adding and editing (or deleting) diet-modification items, and several of their related user-interface elements are shown in FIGS. 10A-10D. FIG. 10A shows a dialog box 135 for adding a modification to the diet, that is displayed by pressing the Add-Modification button 131 of the input form 123 (FIG. 9A). FIG. 10B, shows a dialog box 136 for editing or deleting a listed modification item by clicking the item in area 132 of the diet-setup input form 123 of FIG. 9A. The list box 137 contains available modification options. The expanded view of this list box is shown in FIG. 10C. The desired quantity of a selection made in list box 137 is specified in list box 138, the expanded view of which is shown in FIG. 10D. List box 138 contains predefined values, but a value can be manually specified by selecting “Other” from the list. This displays an input box 139 that allows the user to specify a value different from those listed. Pressing the Add button 140 of the dialog box 135, closes the dialog box and creates a new diet modification that becomes included in the item list 132 of the diet-setup input form 123. Pressing the Cancel button 141 of this dialog box discards all changes and closes the dialog box.

Pressing the Save button 142 of the dialog box 136 of FIG. 10B, closes the dialog box and updates the characteristics (type and amount) of the diet-modification item that was selected for editing in the diet-setup input form 123. Pressing the Delete button 143 closes the dialog box and removes the modification item that was being edited.

It should be noted that the system 100 of the current invention optionally includes in its various embodiments, without loss of generality, the contents of list box 137 and 138 (or any of its alternative implementations thereof), may include other entries in number and order from those presented here.

In further embodiments, the simplified dialog boxes of FIG. 10A, and FIG. 10B may preferably be replaced by expanded versions that gives the user more flexibility in specifying input data types. Additional data types may preferably include (but not limited to the following): (1) Nutrient quantity: calorie, mass etc.; (2) Numeric value type: single-valued, range, lower/upper nutrient limits etc.; (3) Units of measure: grams, milligrams, percent etc.

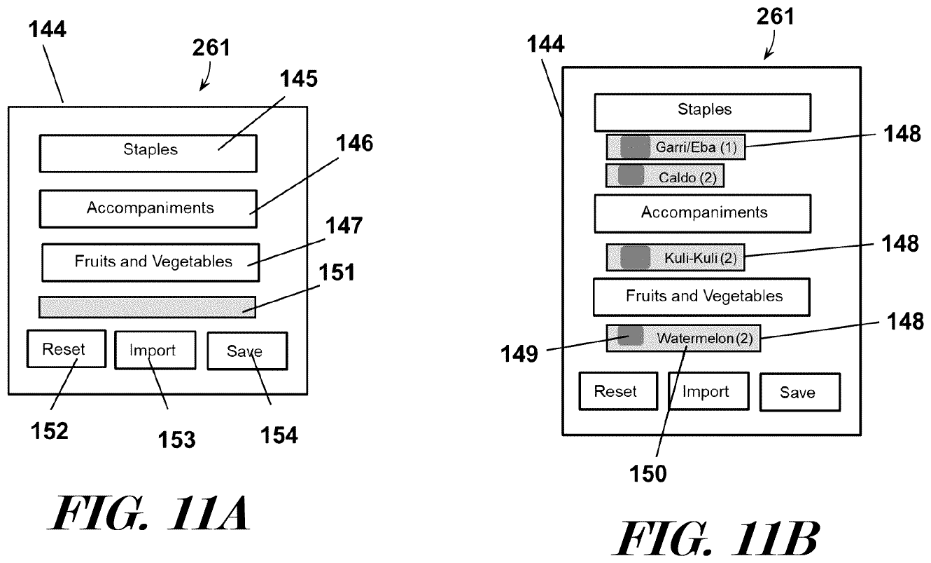

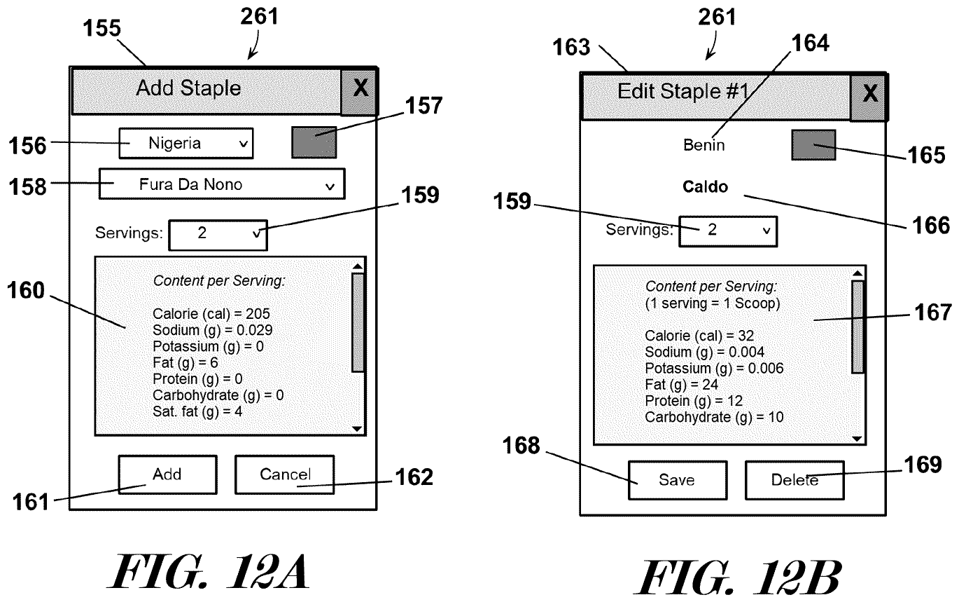

An example dish-setup form 144 for native food access for native food item data records 223A is shown in FIGS. 11A and 11B in which FIG. 11A shows the empty form without any food items defined and FIG. 11B shows the form with some food items defined. We recall from above that native food refers to the main geographical food context of a user 101 of the system 100. User interface features for accessing custom food choices from restaurants and similar food vendor users 101B, are discussed later below. For illustrative purposes, and without loss of generality, several views and dialog boxes related to the native form, that are displayed here, portray data drawn from the example of a West-African food source. Suitable food categories applicable to this geographical region are “Staples,” “Accompaniments,” and “Fruits and Vegetables.”

Dish-setup form 144 of the graphical user interface 261 is the input area associated with the Dish-Setup button 121 of the user pane 119 of the Vertical main-view layout (see FIG. 5A) as well as of the input area 204 of the Stepper layout (FIG. 6B). A dish is a collection of food items. FIG. 11A shows a form when the dish is empty, while FIG. 11B shows a form after several food items have been added. The dish-setup form 144 contains buttons for adding food items belonging to predefined food categories. These are the Staples button 145, the Accompaniments button 146, and the Fruits and Vegetables button 147. Pressing each of these buttons opens a dialog box for selecting a food item of the appropriate category. In FIG. 11B the dish food items 148 are listed below the corresponding buttons of the food categories they belong to. Each food item may preferably include a country flag icon 149 and the food name 150, with its specified number of servings enclosed in parenthesis. Clicking any of the listed dish food items 148 opens a dialog box for editing it (changing the number of its servings). This dialog box may also be used to delete the dish food item 148. Dish error messages during a calculation are displayed in the area 151. This area 151 is hidden if there are no dish errors.

Pressing the Reset button 152, resets (empties) the dish-creation form of all its food items. The Import button 153 displays a dialog box (not shown) for importing and merging with the current dish, dishes previously saved to the user's local food library (which may include dishes containing custom food items). The Save button 154 displays a dialog box (not shown) for saving the current native dish data record 225A to the user's food library 229. The dialog box includes a provision for entering an identifying name input for the dish of the native dish data record 225A being saved.

Various example views related to dialog boxes for adding and editing a staple food item data record 223 are shown in FIGS. 12A and 12B in which FIG. 12A shows a dialog box for adding a new staple food item data record 223 to the dish data record 225 and FIG. 12B shows a dialog box for editing a pre-existing staple food item data record 223 and for deleting it.

The editor form is also used to remove a food item data record 223. Pressing the Staples button 145 of FIG. 11, displays the dialog box 155 shown in FIG. 12A for adding a staple food item. The food's country of origin is selected from list box 156. A flag icon of the selected country may preferably be displayed at 157. List box 158 contains the available staple foods of the selected country. The number of desired servings can be specified from list box 159, by either selecting one of the pre-defined (numeric) values, or by selecting “Other.” In the latter case an input box is displayed for manually entering a servings number or amount. The scrollable box 160 contains information about the nutrient content per serving data 224 of the selected food item of the food item data record 223. Pressing the Add button 161, adds the selected food item of the food item data record 223 to a selected dish data record 225 and closes the dialog box. Pressing the Cancel button 162, closes the dialog box without any further action being taken. Example expanded views of the list boxes 156, 158 and 159 are shown in FIGS. 10A-10D.

Clicking a dish food item 148 in the dish-setup form 144 of FIGS. 11A and 11B, opens the dialog box 163 for editing (or deleting) the item that is shown in FIG. 12B. Editing an item preferably entails changing its number of servings using the list box 159 which functions in the same way as in FIG. 12A. In the example shown in the figure, 164 is the country of origin of the food, 165 is the country's flag icon, and 166 is the food's name. The food's nutritional data per serving is displayed in the scrollable box 167. Pressing the Save button 168, closes the dialog box and updates any changes to the number of servings of the food item being edited. Pressing the Delete button 169 closes the dialog box and removes the food item from the dish-setup form 144 of FIGS. 11A and 11B.

Similar dialog boxes as above (but differing only in captions and the category of food items) are preferably used for adding Accompaniments and Fruits and Vegetables to the dish data record 225.

In other embodiments, a “favorite list” feature may be incorporated in these dialog boxes, in the Dish-setup form 144, or other suitable area(s) of the system 100. This list will be a collection of the most-frequently-selected food item data record 223 by the dietary user 101A over an extended period of use of the system 100. Furthermore, a search-box feature may be provided that allows a dietary user 101A to find food items by typing in a search term. Recommendations of new food choices may also be presented to the dietary user 101A (rendered appropriately in any number of ways), using machine-learning techniques that are based on the dietary user's 101A food choice history.

A user 101 may create a dish data record 225 comprising custom food offerings of custom food item data records 223A from a food vendor user 101B, by scanning a unique scan code 228 that may be a graphical code such as a QR code or a barcode provided by the vendor user 101B. Crucially, the unique scan code 228 encodes the appropriate data-retrieval identifier for accessing food data of the system database 220.

Scanning is enabled by the availability of a suitable facility that is adapted for this purpose in the client program 252 on the user's 101 client device 400. Typically, this is a video camera (for mobile devices), or a webcam (for PCs, Macs etc.) operating in scan mode. Without loss of generality, we will assume this to be a video camera in this discussion.

FIGS. 13A-13C show an example of expanded views of list boxes of the dialog boxes of FIG. 12 in which FIG. 13A shows list box 159 for selecting the desired number of food servings; FIG. 13B shows list box 158 for selecting a food-item; and FIG. 13C shows list box 156 for selecting a country of origin of food items.

FIG. 14 illustrates an example of a dialog box used for scanning in custom food data according to various embodiments described herein. Access to custom food data preferably begins by pressing the scan button 126 of the toolbar 118 of FIG. 5A. This opens the docked dialog box 170 shown in FIG. 14, which preferably overlaps the main user pane 119 of the graphical user interface 261. The view area 171 contains the current camera view from which the unique data identifier 236 may be read. The user 101 positions the unique data identifier 236 appropriately under the camera of their client device 400 for scanning, until the unique data identifier 236 is successfully captured and verified. The verification process involves not only making sure that the encoded data is compatible with the system 100, but also confirming that the requisite food data is available in the system database 220. A success message is posted inside view area 171 if the unique data identifier 236 is successfully verified, otherwise a failure message is posted.

The Continue button 172 is initially disabled when the dialog box 170 is opened. It becomes enabled following a successful unique data identifier 236 scan. Pressing this button closes the dialog box and optionally causes the relevant food data to be transferred from the system database 220 to the client device 400. The returned data is formatted as menus of food items to be displayed at the client device 400. Pressing the Cancel button 173 cancels the scan and closes the dialog box.

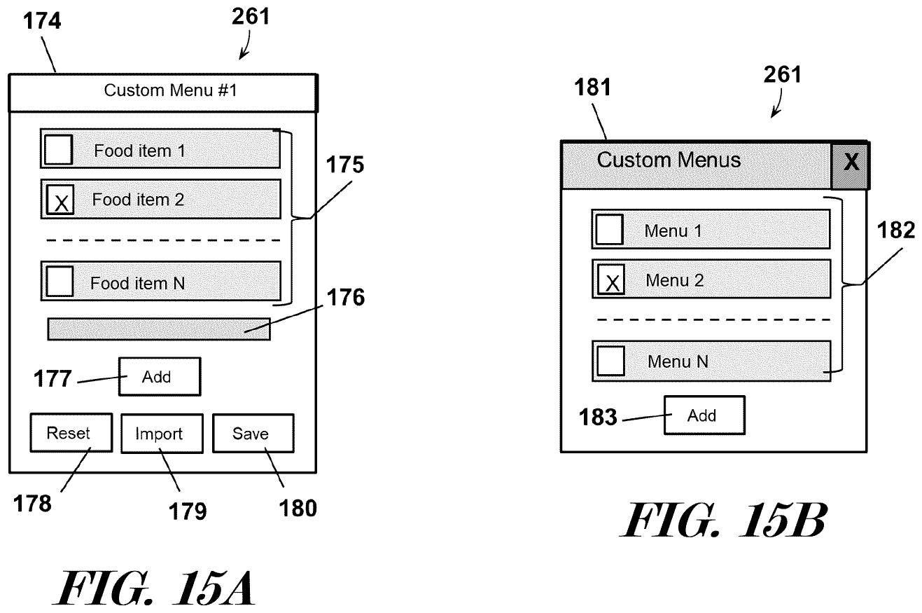

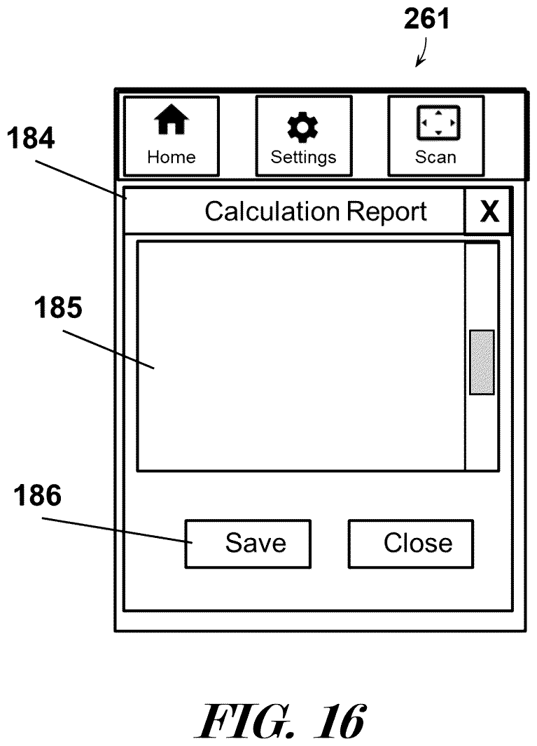

FIGS. 15A and 15B illustrates several example input views of the system 100 which may be used for selecting custom food item data records 225B after a successful data scan in which FIG. 15A shows Dish-Setup custom-menu input form 174 for food inputs; and FIG. 15B shows custom-menu selection dialog box 181. The way the scanned custom food data is displayed on the display screen 404A by the graphical user interface 261 depends on the number of downloaded food menus. These are illustrated in FIGS. 15A and 15B. For a single menu download, the dish-setup form 144 of FIGS. 11A and 11B is now occupied by a Dish-Setup custom-menu input form 174 with the structure depicted in FIG. 16A. Available food items may preferably be presented as a check-box list of items 175. The user 101 may specify food item data records 223 of interest by selecting their corresponding check boxes. Clicking a food item (not the check box) of this list opens a dialog box (not shown) that displays nutrient content per serving data 224 of the food item. Dish error messages (due to interrupted calculations) may be displayed in the area 176. This area is hidden if there are no error messages. The Add button 177 is preferably disabled for this case of a single menu. However, it is enabled when there are multiple menus on display as discussed below. Pressing the Reset button 178, resets (un-checks) all the food items of the form. The Import button 179 displays a dialog box (not shown) for importing and merging with the current dish, dishes previously saved to the user's food library (which may include dishes containing native food items). The Save button 180 displays a dialog box (not shown) that enables the user 101 to save the current dish data record 225 to the user's dish library 232.

For the case of two or more downloaded menus, a dialog box 181 such as that illustrated by FIG. 15B, may be initially opened after a successful scan process. In this dialog box available menus are presented as items 182 of a check-box list. A user 101 may then select menus of interest by checking the relevant checkboxes. Pressing the Add button 183 (which is disabled if no selection is made) opens the dish-menu input area using an input form like that shown in FIG. 15A with a merged view of the food items of the selected menus, and with the Add button 177 now enabled. Pressing this button 177 again reopens dialog box 181.

We now preface the discussion of the calculation process by first reviewing some important relevant considerations based on FDA guidelines.

A daily total calorie intake of 2000 calories (cal.) is the FDA recommended value for general nutritional advice of the average healthy adult. This is also the default target calorie value of this invention. However, the user 101 may override this by entering a different value. The FDA provides a table of recommended Daily Values (DV) of nutrients which is partially reproduced in Table 1. Thus, as an example, the maximum daily protein intake of a healthy adult is recommended to not exceed 50 grams (g). Related to the DVs are the Percent Daily Values (% DV), which expresses a given quantity of nutrient as a fraction (in percent) of its corresponding DV. Expressing values in percentages provides for a convenient and uniform way of keeping track of nutrient intake. This is an important metric that is commonly listed for all nutrients in nutritional food labels. Thus, continuing with the above protein example, a person who initially consumes 10 g of protein, consumes 20% DV, with a balance of 80% DV to go for the day.

The calculation algorithms accounts for additional FDA information and recommendations as follows: (1) Carbohydrates provide 4 calories per gram, protein provides 4 calories per gram, and fat (including saturated fat, trans fat and cholesterol) provides 9 calories per gram.; (2) 5% DV or less of a nutrient per serving is considered low; (3) 20% DV or more of a nutrient per serving is considered high.

TABLE 1
FDA recommended daily values of food nutrients
for an average healthy adult.

| Nutrient | Daily Value (DV) | |
|---|---|---|
| Calcium | 1300 | mg |
| Fat | 78 | g |
| Phosphorus | 1250 | mg |
| Potassium | 4700 | mg |
| Sodium | 2300 | mg |
| Carbohydrate | 275 | g |
| Cholesterol | 300 | mg |
| Iron | 18 | mg |
| Protein | 50 | g |
| Saturated fat | 20 | g |
| Added sugars | 50 | g |

The calculation process preferably invokes a general-purpose solver of a client program 252 for handling the various target diet choices a user 101 can make. A solver is a function that uses a specific adaptable algorithm to generate output appropriate for the target diet of a target diet data record 221A selected by a user 101.

The essential task of a solver is tallying those nutritional characteristics of the user's dish components that are relevant to the specified target diet. It then produces an output (Calculation Report Outputs of Table 2 and Table 3) that describes how the nutritional characteristics data 226 of a dish data record 225 compares with the data of the recommended nutritional objective data record 222A of the target diet data record 221A, the output preferably including (but not limited) to the following two parts: (1) A comparative analysis of the tally results against target diet properties, with met and unmet dietary goals highlighted, and with recommended nutritional objective that are corrective recommendations in the latter case, to bring it to within the constraints of the target; (2) Tabulation of the full breakdown of the nutritional properties of the dish components, highlighting those components contributing to the dietary goals.

In further embodiments, the output that describes how the nutritional characteristics data 226 of the dish data record 225 compares with recommended nutritional objectives data 222A of the target diet data record 221A may comprise data describing how at least one nutritional characteristic of the dish data record 225 does not meet at least one recommended nutritional objective of the target diet data record 221A. Referring to the example of Table 2, data describing how at least one nutritional characteristic of the dish data record 225 does not meet at least one recommended nutritional objective of the target diet data record 221A includes: “1 servings of Jollof Rice accounts for 40% of your current calorie intake, and 61% of your protein intake.”; “2 servings of Kontomire stew accounts for 46% of your current calorie intake, and 33% of your protein intake.”; and “2 servings of Pear accounts for 14% of your current calorie intake, and 6% of your protein intake.” Referring to the example of Table 3, data describing how at least one nutritional characteristic of the dish data record 225 does not meet at least one recommended nutritional objective of the target diet data record 221A includes: “1 serving of Jollof Rice accounts for 99% of your cholesterol intake.”; “2 servings of Kontomire stew accounts for 1% of your current cholesterol intake.”; and “2 servings of Pear do not contribute to your cholesterol intake.”

In further embodiments, the output may comprise a corrective dish modification that may be displayed on the display screen 404A of the dietary client device 400A. Referring to the example of Table 2, corrective dish modifications that may be displayed include: “Target Calorie: Off target! Add at least 392 cal to your dish.”; “Carbohydrates: Off target! Reduce your carbs by at least 24%”; and “Protein: Off target! Reduce your protein by at least 10%”. Referring to the example of Table 3, corrective dish modifications that may be displayed include: “Target Calorie: Off target! Reduce your dish by at least 38 cal.”; and “The total cholesterol content of your dish (32.022 g) is higher than the FDA recommended limit of 0.15 g.” Optionally, the output may include information about how the dish of the dish data record 225 conforms with the FDA daily diet recommendations.

FIG. 16 illustrates an example of a dialog box for displaying the docked Calculation Report view dialog box 184 according to various embodiments described herein. Upon completion of the calculation, the output is presented in a window (Calculation Report view dialog box 184) (FIG. 16), which overlaps the user pane 119 of FIG. 5A. The calculation output is displayed in the scrollable box 185. The Save button 186 opens a dialog box (not shown) for saving the current calculation data to the user's calculation library. The dialog box includes a provision for entering an identifying name input for the calculation being saved.

We conclude our discussion of calculations by considering two simple examples of the calculation diabetic and heart-healthy target diets 221A using the same dish input.

The nutritional goals of a diabetic diet data record 221 may be summarized thus: “Carbohydrates should constitute 50%-60% of total calories. Proteins 15%-20% dietary intake.”

Sample user 101 inputs via graphical user interface 261 and the resulting calculation results are summarized in Table 2. For brevity only the food nutritional content that are germane to this example are included in Table 2.

For the diabetic diet data record 221 that the user 101 has selected as their target diet data record 221A, the solver of the client program 252 uses an algorithm centered around well-defined goals as enumerated in Table 2. The process of succinctly identifying and expressing the diet goals in suitable mathematical forms is paramount in the implementation of the solver. The rest of the calculation involves the application of relatively simple algebraic logic and data manipulation in a way that should readily be obvious to persons well versed in the arts.

A heart-healthy diet data record 221 that the user 101 has selected as their target diet data record 221A for its part has the rather brief nutritional goal: “A low cholesterol diet.” A summary of the calculation results of this heart-healthy diet example is given in Table 3, whereas before, only the relevant nutritional food contents are shown. To meet the stated low-cholesterol goal, an upper (limiting) bound of the cholesterol content is preferably defined in consonant with the previously-referenced FDA recommendation that 5% DV or less of a nutrient per serving is considered low for a healthy adult. Since from Table 3, the DV for cholesterol is 300 mg, the required limiting value will preferably be 15 mg. The total cholesterol content of the dish of the dish data record 225 preferably should not exceed this value to satisfy the constraints of the target diet. As before, the calculation logic involved is relatively simple and should be readily obvious to persons well versed in the arts.

The calculation outputs may optionally be listed verbally and/or displayed via a GUI 261 as shown in the Tables 2 and 3. However, in other embodiments, the system 100 may express calculation outputs in other ways including using charts and other graphical elements. Additionally, other related post-calculation features preferably may be included with the output, such as other means of suggesting alternative food servings to satisfy the unmet diet objectives. This covers other approaches such as the use of interactive data input wizards, gaming methods etc.

TABLE 2
A summary of diabetic diet and dish inputs and salient calculation
output for an illustrative example described in the text.
Diet

| Type | Goals | | Calorie (cal) | Modifications |
|---|---|---|---|---|
| Diabetic | (1) | Carbohydrates: 50%-60% | 1700 | — |
| | | of total calories. | | |
| | (2) | Proteins: 15%-20% | | |
| | | dietary intake. | | |

Dish

| | | | Calorie | Carbohydrate | Protein |
|---|---|---|---|---|---|
| Name (country) | | Servings | (cal) | (g) | (g) |
| Staples | Jollof Rice | 1 | 284 | 33 | 13 |
| | (Nigeria) | | | | |
| Accompa- | Kontomire | 2 | 325 | 31 | 7 |
| niments | stew | | | | |
| | (Ghana) | | | | |
| Fruits and | Pear | 2 | 102 | 27 | 1 |
| vegetables | (Cameroon) | | | | |

Calculation Report Output

| | |
|---|---|
| Global | Target Calorie: Off target! Add at least 392 cal to your dish. |
| | Carbohydrates: Off target! Reduce your carbs by at least 24% |
| | Protein: Off target! Reduce your protein by at least 10% |
| Staples | 1 serving of Jollof Rice accounts for 40% of your current |
| | calorie intake, and 61% of your protein intake. |
| Accompa- | 2 servings of Kontomire stew accounts for 46% of your |
| niments | current calorie intake, and 33% of your protein intake. |
| Fruits and | 2 servings of Pear accounts for 14% of your current calorie |
| vegetables | intake, and 6% of your protein intake. |

TABLE 3
A summary of heart-healthy diet and dish inputs and salient calculation
output for an illustrative example described in the text.
Diet

| Type | Goals | Calorie (cal) | Modifications |
|---|---|---|---|
| Diabetic | The total cholesterol of dish | 1000 | — |
| | is not to exceed 0.15 g. | | |

Dish

| | | | Calorie/ | Cholesterol/ |
|---|---|---|---|---|
| Name (country) | | Servings | serving (cal) | serving (g) |
| Staples | Jollof Rice | 1 | 284 | 33 |
| | (Nigeria) | | | |
| Accompa- | Kontomire | 2 | 325 | 0.011 |
| niments | stew | | | |
| | (Ghana) | | | |
| Fruits and | Pear | 2 | 102 | 0 |
| vegetables | (Cameroon) | | | |

Calculation Report Output

| | |
|---|---|
| Global | Target Calorie: Off target! Reduce your dish by at least 38 |
| | cal. The total cholesterol content of your dish (32.022 g) |
| | is higher than the FDA recommended limit of 0.15 g. |
| | Select food items with lower cholesterol content. |
| Staples | 1 serving of Jollof Rice accounts for 99% of your |
| | cholesterol intake. |
| Accompa- | 2 servings of Kontomire stew accounts for 1% of your |
| niments | current cholesterol intake. |
| Fruits and | 2 servings of Pear do not contribute to your cholesterol |
| vegetables | intake. |

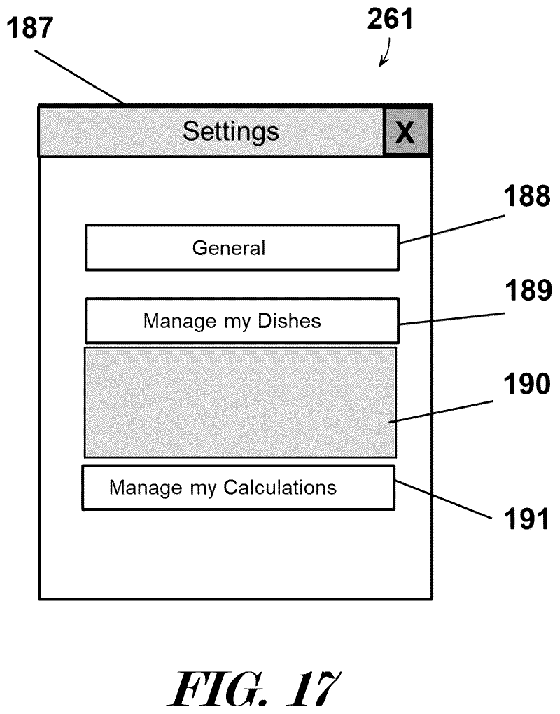

Pressing the Settings button 125 of the main view (FIG. 5A) of the graphical user interface 261, opens a docked dialog box that overlaps the user pane 119. An illustration of an example of this the Settings view dialog box 187 is shown in FIG. 17, in which three drop-down buttons (188, 189 and 191) are shown for accessing additional views and functionalities related to the buttons. When expanded the drop-down buttons 188, 189,191 display views (forms or docked dialog boxes) right below them (for example in the FIG. 17 the drop-down button 189 is depicted in its expanded state, and its associated input form will be displayed in the area 190). The system 100 may include other embodiments, in which other functionalities (besides the three shown here) and including custom-diet management functionality (not shown), may be available, and in which alternative design concepts may be employed.

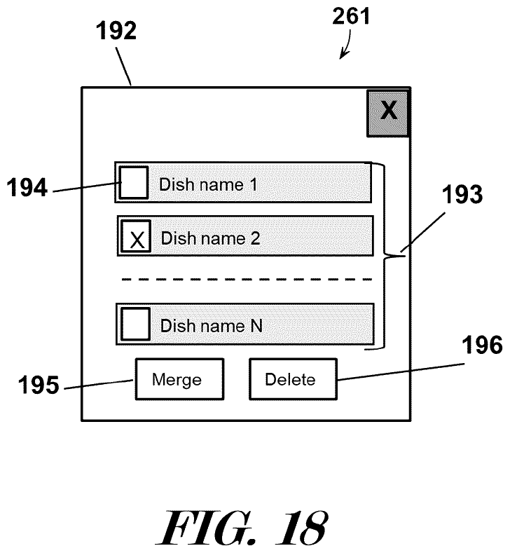

Previously stored dish data records 225 by the user 101 are accessible and managed by pressing and expanding the Manage my Dishes button 189. This displays the docket form or dialog box 192 shown in FIG. 18. The dialog box 192 may preferably include a check-box list 193 of (the identifying names of) dishes stored in the dish library. If this list is empty, a message to that effect is displayed instead in place of the dish list.

The user 101 can select dish data records 225 of interest by selecting their checkboxes 194, and then taking further action by pressing the Merge button 195 or the Delete button 196. Pressing the Merge button 195 merges the food items of the selected dishes into the dish data record 225 of the current session, and the user 101 is then navigated to the expanded Dish-setup view (of FIG. 11). Pressing the Delete button 196 removes the selected dish items. Clicking a dish item (not the check box) opens a dialog box (not shown) that displays the food content or food item(s) 223 of the dish data record 225.

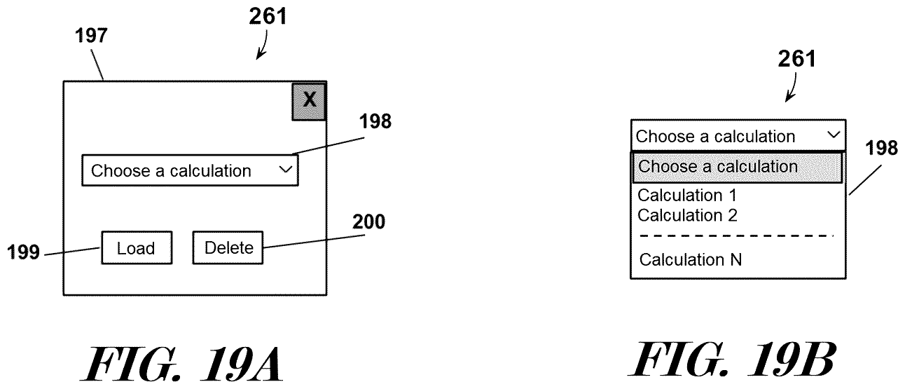

Pressing and expanding the Manage my Calculations button 191 of FIG. 17, displays the dialog 197 shown in FIG. 19. An example of the whole form is shown in FIG. 19A and may include a, preferably drop-down, calculation-selection list box 198 of (the identifying names of) calculation items stored in the calculation library 230. If this calculation-selection list box 198 is empty, a message to that effect is displayed in its place instead. An expanded view of this calculation-selection list box 198 is depicted in FIG. 19B. A calculation item encapsulates all the associated data (diet, dish, and calculation output) of a prior successfully calculated session.

After selecting an item from calculation-selection list box 198, a user 101 can load it into the current session by pressing the Load button 199 or remove it from the calculate library 230 by pressing the Delete button 200. If the Load button 199 is pressed, the calculation is loaded in to become the current session, and the user 101 is then navigated to the expanded Calculation Report view dialog box 184 of FIG. 16 of graphical user interface 261. All the diet and dish data corresponding to the loaded calculation may also be accessed from the other data areas of the main view (FIG. 11).

The General button 188 of FIG. 17 may be used to access general (“catch-all”) settings of the client device 400 of the user 101. Its expanded state form area (not shown) may be used to provide other means for the user to specify client device 400/system 100 settings not covered above. These may include for example means to change the color scheme and other display characteristics of the graphical user interface 261, to specify a display language for the client device 400 (English, French etc.), to specify a user 101 time zone to facilitate time-sensitive messaging to the user as needed, and to set other user configuration data 233 that may be relevant.

FIG. 18 illustrates an example of a dialog box 192 managing the user's dish library 232 according to various embodiments described herein.

FIGS. 19A and 19B illustrate an example of a dialog box 197 managing the user's calculation library 230 in which FIG. 19A shows the whole dialog box 197 form and 19B shows expanded view of the calculation-selection list box 198.

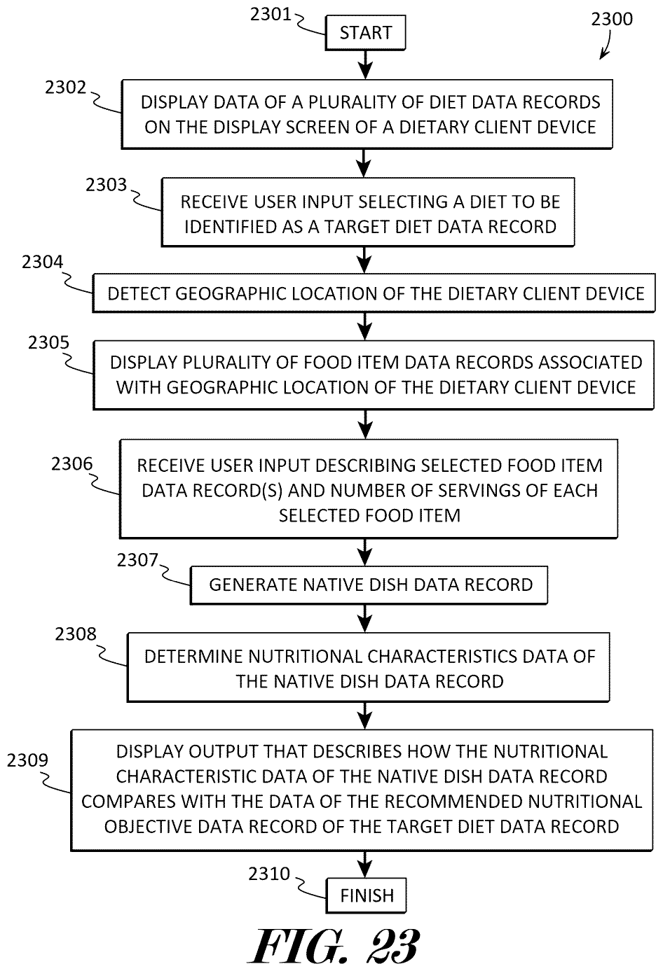

FIG. 23 depicts a block diagram of an example of a computer-implemented method of generating a system output that describes how nutritional characteristic data of a native dish data record 225A compares with data of a recommended nutritional objective data record 222A of a target diet data record 221A (“method 2300”) according to various embodiments described herein. Program instructions, such as contained in a system management program 251 and a client program 252 and stored in one or more computer-readable memories 310, 410, may be executed via one or more processors 302, 402, to cause the system 100 to perform the steps of method 2300.

The method 2300 may start 2301, and the system 100 may display data of a plurality of diet data records 221 on the display screen 404A of a dietary client device 400A in step 2302. In preferred embodiments, a client program 252 may generate a graphical user interface 261 on the display screen 404A of the dietary client device 400A, and the graphical user interface 261 may display the data of a plurality of diet data records on the display screen 404A of the dietary client device 400A. Preferably, each diet data record 221 of the plurality of diet data records 221 may be associated with a recommended nutritional objectives data record 222 in the system database 220. Optionally, all or parts of the system database 220 may be stored in a data store 308 of a system server 300A, and the dietary client device 400A may be one of a plurality of dietary client devices 400A that are in network communication with the system server 300A. Optionally, all or parts of the system database 220 may be stored in a data store 408 of the dietary client device 400A.

In step 2303, the system 100 may receive user input selecting a diet that is to be identified as a target diet data record 221A. In preferred embodiments, the dietary user 101A may interact with the graphical user interface 261, and the client program 252 may generate the user input based on a selection the dietary user 101A makes using a Diet-Setup button 120 or any other suitable selection method which may allow a dietary user 101A to select one of the diet data records 221 as a target diet data record 221A.

In some embodiments of step 2303, the client program 252 may display, via the GUI 261 of the dietary client device 400A, a diet-setup input form 123, and the client program 252 may receive, via the GUI 261 of the dietary client device 400A, user input that modifies a recommended nutritional objective of the recommended nutritional objectives data 222A of the target diet data record 221A. For example, the GUI 261 may display a diet-setup input form 123 and related views by which the dietary user 101A is able to select a target diet 221A and make modifications to its set of recommended nutritional objectives 222A e.g., calorie value and its other nutritional properties.

In step 2304, the system 100 may detect the geographic location of the dietary client device 400A. In preferred embodiments, a client program 252 may use global positioning system (GPS), network location data, or other location detection functions of the dietary client device 400A to determine a geographic location of the dietary client device 400A

In step 2305, the system 100 may display, via the display screen 404A of the dietary client device 400A, a plurality of food item data records 223 (which may include native food item data records 223A and/or custom food item data records 223B) associated with the geographic location of the dietary client device 400A. In preferred embodiments, a system management program 251 and/or a client program 252 may compare the geographic location of the dietary client device 400A to the location data 237 associated with food item data records 223 in the system database 220 to determine which food item data records 223 to display on the GUI 261. For example, if the geographic location of the dietary client device 400A is determined to be in West-Africa, food item data records 223 associated with the geographic location of West-Africa may be displayed on the GUI 261.

In step 2306, the system 100 may receive user input describing one or more selected native food item data record(s) 223A and a number of servings of each selected native food item of the selected native food item data record(s) 223A. In preferred embodiments, the dietary user 101A may interact with the graphical user interface 261, and the client program 252 may generate the user input based on a selection the dietary user 101A makes using a Dish-setup form 144 and Dish-Setup button 121 in which each food item data record 223 may preferably include the food name 150, with its specified number of servings enclosed in parenthesis. Clicking any of the listed dish food items 148 opens a dialog box for editing it (changing the number of its servings). In further embodiments, any other suitable selection method which may allow a dietary user 101A to select one or more of the food item data records 223 and a number of servings of each selected food item may be used.

In step 2307, the system 100 may generate a native dish data record 225A in the system database 220. In preferred embodiments, a system management program 251 and/or a client program 252 may generate the native dish data record 225A in the system database 220 in which the native dish data record 225A includes the data of the one or more selected native food item data records 223A and the number of servings of each of the one or more selected native food item data records 223A.

In step 2308, the system 100 may determine nutritional characteristics data 226 of the native dish data record 225A. Each native dish data record 225A may comprise or be associated with one or more food item data records 223 and the nutrient content per serving data 224 of the one or more food item data records 223. Each native dish data record 225A may also comprise or be associated with nutritional characteristics data 226A which may describe the number of servings of each food item in the dish and the total of all the nutrient content per serving data 224 of the one or more food items in the dish. Native dish data records 225A may comprise data of at least one native food item data record 223A, and optionally may comprise data of one or more custom food item data records 223B input by a vendor user 101B. Preferably, a system management program 251 and/or a client program 252 may use the number of servings of each food item in the dish and the total of all the nutrient content per serving data 224 of the one or more food items in the dish to determine the nutritional characteristics data 226A. In some embodiments, step 2308 may be performed by a processor 402 of the dietary client device 400A that may be executing the program instructions of the client program 252. In some embodiments, step 2308 may be performed by a processor 302 of a system server 300A that may be executing the program instructions of the system management program 251.

In step 2309, the system 100 may display an output that describes how the nutritional characteristic data 226A of the native dish data record 225B compares with the data of the recommended nutritional objective data record 222A of the target diet data record 221A. In preferred embodiments, a client program 252, preferably executed by a processor 402 of the dietary client device 400A, may generate and display, via the GUI 261 on the display screen 404A of the dietary client device 400A, an output that describes how the nutritional characteristic data 226A of the native dish data record 225B compares with the data of the recommended nutritional objective data record 222A of the target diet data record 221A. In further preferred embodiments, the client program 252 may comprise a solver configured for tallying those nutritional characteristics of the user's dish components that are relevant to the specified target diet. The client program 252 then produces an output that describes how the nutritional characteristic data 226 of a dish data record 225 compares with the data of the recommended nutritional objective data record 222A of the target diet data record 221A, the output preferably including (but not limited) to the following two parts: (1) A comparative analysis of the tally results against target diet properties, with met and unmet dietary goals highlighted, and with recommended nutritional objective(s) that are corrective recommendations in the latter case, to bring it to within the constraints of the target; (2) Tabulation of the full breakdown of the nutritional properties of the dish components, highlighting those components contributing to the dietary goals.

In further embodiments, the output that describes how the nutritional characteristics data 226 of the dish data record 225 compares with recommended nutritional objectives data 222A of the target diet data record 221A may comprise data describing how at least one nutritional characteristic of the dish data record 225 does not meet at least one recommended nutritional objective of the target diet data record 221A.

In further embodiments, the output may comprise a corrective dish modification that may be displayed on the display screen 404A of the dietary client device 400A.

After step 2309, method 2300 may finish 2310.

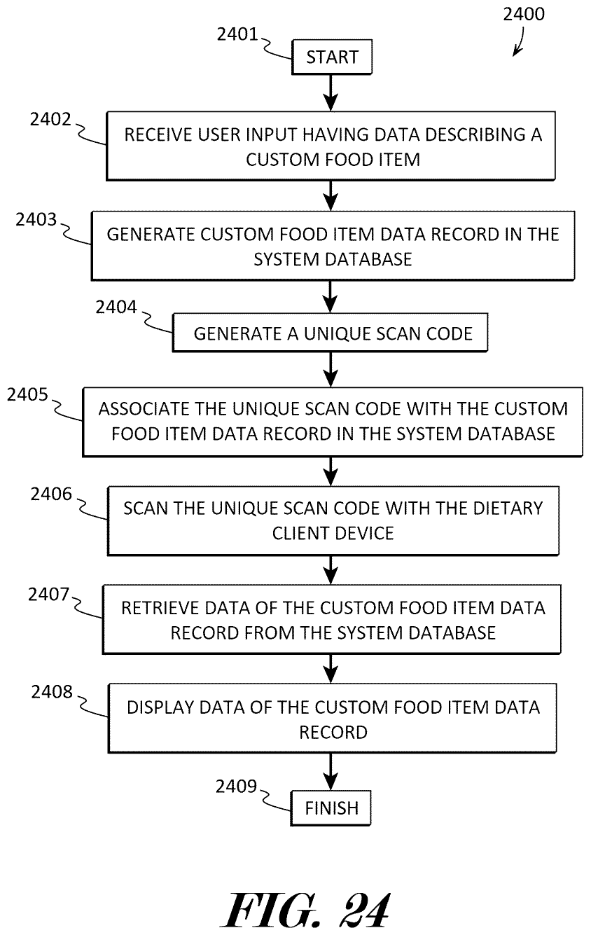

FIG. 24 illustrates a block diagram of an example of a computer-implemented method of providing custom food item data to a client device 400 (“method 2400”) according to various embodiments described herein. Program instructions, such as contained in a system management program 251 and a client program 252 and stored in one or more computer-readable memories 310, 410, may be executed via one or more processors 302, 402, to cause the system 100 to perform the steps of method 2400.

The method 2400 may start 2401, and the system 100 may receive user input having data describing a custom food item in step 2402. In preferred embodiments, a vendor user 101B may interact with a graphical user interface 261 generated by a client program 252 on their vendor client device 400B to provide user input having data describing a custom food item, in which the data describing the custom food item includes nutrient content per serving data of the custom food item. The data describing the custom food item may include the name of the custom food item and other descriptive information of the custom food item.

In step 2403, the system 100 may generate a custom food item data record 223B in the system database 220. In preferred embodiments, a system management program 251 and/or a client program 252 may generate the custom food item data record 223B in the system database 220, the custom food item data record 223B having the nutrient content per serving data 224B of the custom food item.

In step 2404, the system 100 may generate a unique scan code 228. In preferred embodiments, a system management program 251 may generate the unique scan code 228. A unique scan code 228 may comprise any type of machine readable code, such as a food-vendor barcode, QR code, etc. Preferably, each unique scan code 228 may be associated with a generated unique data identifier 236 that may encode universal resource locator (URL) information that can be retrieved by scan code readers of dietary client devices 400A to facilitate access of dietary user 101A to custom food data of the system 100.

In step 2405, the system 100 may associate the unique scan code 228 with the custom food item data record 223B in the system database 220. In preferred embodiments, a system management program 251 may associate the unique scan code 228 with the custom food item data record 223B in the system database 220.

In step 2406, the unique scan code 228 may be scanned with a dietary client device 400A. In preferred embodiments, a camera type of I/O interface 404 of a dietary client device 400A may record a physical representation of the unique scan code 228 which may allow a client program 252 running on the dietary client device 400A to read the unique scan code 228.

In step 2407, data of the custom food item data record 223B associated with the unique scan code 228 may be retrieved from the system database 220 by the system 100. In preferred embodiments, a client program 252 and/or a system management program 251 may retrieve the data of the custom food item data record 223B associated with the unique scan code 228 from the system database 220.

In step 2408, the system 100 may display data of the custom food item data record 223B on the display screen 404A of the dietary client device 400A. In preferred embodiments, a client program 252 may display data of the custom food item data record 223B via a GUI 261 on the display screen 404A of the dietary client device 400A.

After step 2408, method 2400 may finish 2409.

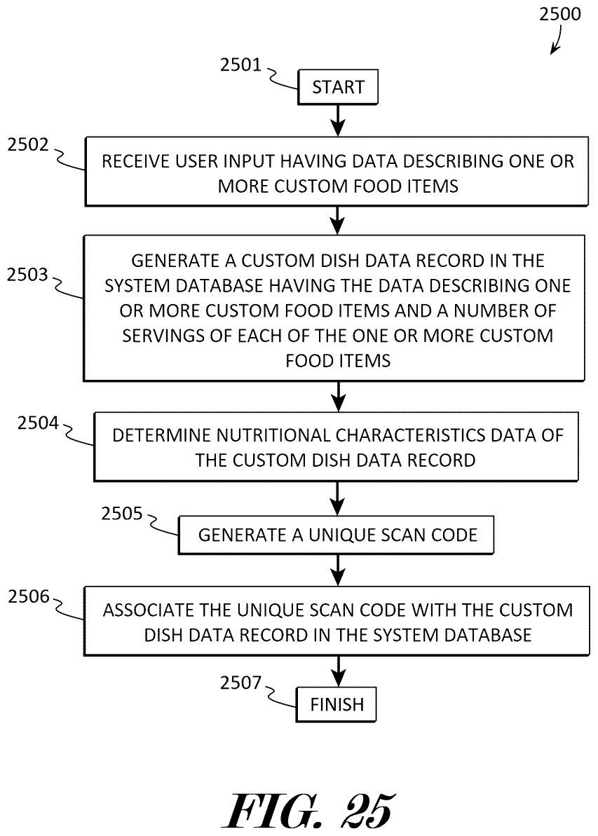

FIG. 25 illustrates a block diagram of an example of a computer-implemented method of generating a custom dish data record 225B (“method 2500”) according to various embodiments described herein. Program instructions, such as contained in a system management program 251 and a client program 252 and stored in one or more computer-readable memories 310, 410, may be executed via one or more processors 302, 402, to cause the system 100 to perform the steps of method 2500.

The method 2500 may start 2501, and the system 100 may receive user input having data describing one or more custom food items in step 2502. In preferred embodiments, a vendor user 101B may interact with a graphical user interface 261 generated by a client program 252 on their vendor client device 400B to provide user input having data describing one or more custom food items, in which the data describing each custom food item includes nutrient content per serving data of the custom food item. The data describing each custom food item may include the name of the custom food item and other descriptive information of the custom food item.

In step 2503, the system may generate a custom dish data record 225B in the system database 220 having the data describing one or more custom food items and a number of servings of each of the one or more custom food items. In preferred embodiments, a system management program 251 and/or a client program 252 may generate the custom dish data record 225B in the system database 220, the custom dish data record 225B having the nutrient content per serving data 224B of each custom food item and a number of servings of each custom food item.

In step 2504, the system 100 may determine nutritional characteristics data 226B of the custom dish data record 225B. Preferably, a system management program 251 and/or a client program 252 may use the number of servings of each food item in the dish and the total of all the nutrient content per serving data 224B of the one or more food items in the dish to determine the nutritional characteristics data 226B. In some embodiments, step 2308 may be performed by a processor 402 of the dietary client device 400A that may be executing the program instructions of the client program 252. In some embodiments, step 2308 may be performed by a processor 302 of a system server 300A that may be executing the program instructions of the system management program 251.

In step 2505, the system 100 may generate a unique scan code 228. In preferred embodiments, a system management program 251 may generate the unique scan code 228. A unique scan code 228 may comprise any type of machine readable code, such as a food-vendor barcode, QR code, etc. Preferably, each unique scan code 228 may be associated with a generated unique data identifier 236 that may encode universal resource locator (URL) information that can be retrieved by scan code readers of dietary client devices 400A to facilitate access of dietary user 101A to custom food data of the system 100.

In step 2506, the system 100 may associate the unique scan code 228 with the custom dish data record 225B in the system database 220. In preferred embodiments, a system management program 251 may associate the unique scan code 228 with the custom dish data record 225B in the system database 220.

After step 2506, method 2500 may finish 2507.

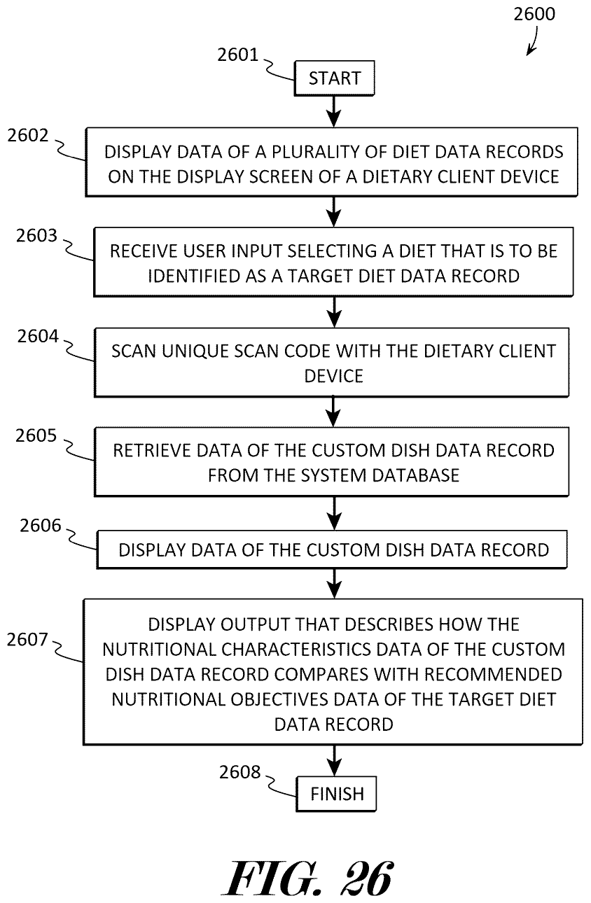

FIG. 26 depicts a block diagram of an example of a computer-implemented method of generating a system output that describes how nutritional characteristic data of a custom dish data record 225B compares with data of a recommended nutritional objective data record 222A of a target diet data record 221A (“method 2600”) according to various embodiments described herein. Program instructions, such as contained in a system management program 251 and a client program 252 and stored in one or more computer-readable memories 310, 410, may be executed via one or more processors 302, 402, to cause the system 100 to perform the steps of method 2600.

The method 2600 may start 2601, and the system 100 may display data of a plurality of diet data records 221 on the display screen 404A of a dietary client device 400A in step 2602. In preferred embodiments, a client program 252 may generate a graphical user interface 261 on the display screen 404A of the dietary client device 400A, and the graphical user interface 261 may display the data of a plurality of diet data records on the display screen 404A of the dietary client device 400A. Preferably, each diet data record 221 of the plurality of diet data records 221 may be associated with a recommended nutritional objectives data record 222 in the system database 220. Optionally, all or parts of the system database 220 may be stored in a data store 308 of a system server 300A, and the dietary client device 400A may be one of a plurality of dietary client devices 400A that are in network communication with the system server 300A. Optionally, all or parts of the system database 220 may be stored in a data store 408 of the dietary client device 400A.

In step 2603, the system 100 may receive user input selecting a diet that is to be identified as a target diet data record 221A. In preferred embodiments, the dietary user 101A may interact with the graphical user interface 261, and the client program 252 may generate the user input based on a selection the dietary user 101A makes using a Diet-Setup button 120 or any other suitable selection method which may allow a dietary user 101A to select one of the diet data records 221 as a target diet data record 221A.

In step 2604, the unique scan code 228 may be scanned with a dietary client device 400A. In preferred embodiments, a camera type of I/O interface 404 of a dietary client device 400A may record a physical representation of the unique scan code 228 which may allow a client program 252 running on the dietary client device 400A to read the unique scan code 228.

In step 2605, data of the custom dish data record 225B associated with the unique scan code 228 may be retrieved from the system database 220 by the system 100. In preferred embodiments, a client program 252 and/or a system management program 251 may retrieve the data of the custom dish data record 225B associated with the unique scan code 228 from the system database 220.

In step 2606, the system 100 may display data of the custom dish data record 225B on the display screen 404A of the dietary client device 400A. In preferred embodiments, a client program 252 may display data of the custom dish data record 225B via a GUI 261 on the display screen 404A of the dietary client device 400A.

In step 2607, the system 100 may display an output that describes how the nutritional characteristic data 226B of the custom dish data record 225B compares with the data of the recommended nutritional objective data record 222A of the target diet data record 221A. In preferred embodiments, a client program 252, preferably executed by a processor 402 of the dietary client device 400A, may generate and display, via the GUI 261 on the display screen 404A of the dietary client device 400A, an output that describes how the nutritional characteristic data 226B of the custom dish data record 225B compares with the data of the recommended nutritional objective data record 222A of the target diet data record 221A. In further preferred embodiments, the client program 252 may comprise a solver configured for tallying those nutritional characteristics of the user's dish components that are relevant to the specified target diet. The client program 252 then produces an output that describes how the nutritional characteristic data 226B of the custom dish data record 225B compares with the data of the recommended nutritional objective data record 222A of the target diet data record 221A, the output preferably including (but not limited) to the following two parts: (1) A comparative analysis of the tally results against target diet properties, with met and unmet dietary goals highlighted, and with recommended nutritional objective(s) that are corrective recommendations in the latter case, to bring it to within the constraints of the target; (2) Tabulation of the full breakdown of the nutritional properties of the dish components, highlighting those components contributing to the dietary goals.

In further embodiments, the output that describes how the nutritional characteristics data 226B of the custom dish data record 225B compares with recommended nutritional objectives data 222A of the target diet data record 221A may comprise data describing how at least one nutritional characteristic of the custom dish data record 225B does not meet at least one recommended nutritional objective of the target diet data record 221A.

In further embodiments, the output may comprise a corrective dish modification that may be displayed on the display screen 404A of the dietary client device 400A.

After step 2607, method 2600 may finish 2608.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine-readable storage device, a machine-readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), light emitting diode (LED) display, or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random-access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD), light emitting diode (LED) display, or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to a processor for execution. For example, the instructions may initially be carried on a magnetic disk of a remote computer. The remote computer can load the instructions for implementing all or a portion of the present invention remotely into a dynamic memory and send the instructions over the air (e.g., through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on a storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetric digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and Wi-Fi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An integrated context-based personal diet management system, the system comprising:
a system database;
a dietary client device having a display screen;
program instructions stored in one or more computer-readable memories; and
one or more processors configured to execute the program instructions to cause the system to perform operations comprising:
generating a graphical user interface (GUI) on the display screen of the dietary client device, wherein the GUI displays data of a plurality of diet data records on the display screen of the dietary client device, and wherein each diet data record of the plurality of diet data records is associated with a recommended nutritional objectives data record in the system database;
receiving, via the dietary client device, user input selecting a diet corresponding to a diet data record of the plurality of diet data records that is to be identified as a target diet data record;
detecting a geographic location of the dietary client device;
displaying, via the GUI on the display screen of the dietary client device, a plurality of native food item data records that are associated with the geographic location of the dietary client device in the system database, wherein each native food item data record of the plurality of native food item data records comprises nutrient content per serving data;
receiving, via the dietary client device, user input having data describing one or more selected native food item data records of the plurality of native food item data records and a number of servings of each of the one or more selected native food item data records;
generating a native dish data record in the system database having data of the one or more selected native food item data records and the number of servings of each of the one or more selected native food item data records;
determining nutritional characteristics data of the native dish data record; and
displaying, via the GUI on the display screen of the dietary client device, an output that describes how the nutritional characteristic data of the native dish data record compares with the data of the recommended nutritional objective data record of the target diet data record.

2. The system of claim 1, wherein the output that describes how the nutritional characteristics data of the native dish data record compares with recommended nutritional objectives data of the target diet data record comprises data describing how at least one nutritional characteristic of the native dish data record does not meet at least one recommended nutritional objective of the target diet data record.

3. The system of claim 2, wherein the one or more processors are configured to execute the program instructions to cause data of a corrective dish modification to be displayed on the display screen of the dietary client device.

4. The system of claim 1, wherein the system database is stored in a data store of a system server, and wherein the dietary client device is one of a plurality of dietary client devices that are in network communication with the system server.

5. The system of claim 1, wherein the system database is stored in a data store of the dietary client device.

6. The system of claim 1, wherein the steps of: determining nutritional characteristics data of the native dish data record; and displaying, via the GUI on the display screen of the dietary client device, an output that describes how the nutritional characteristic data of the native dish data record compares with the data of the recommended nutritional objective data record of the target diet data record are both performed via a processor of the dietary client device.

7. The system of claim 1, wherein the one or more processors are configured to execute the program instructions to cause the system to perform operations comprising: displaying, via the GUI of the dietary client device, a diet-setup input form; and receiving, via the GUI of the dietary client device, user input that modifies a recommended nutritional objective of the target diet data record.

8. The system of claim 1, further comprising a vendor client device that is in network communication with a system server.

9. The system of claim 8, wherein the one or more processors are configured to execute the program instructions to cause the system to perform operations comprising: receiving, via the vendor client device, user input having data describing a custom food item, wherein the data describing the custom food item includes nutrient content per serving data of the custom food item; and generating a custom food item data record in the system database having the nutrient content per serving data of the custom food item.

10. The system of claim 9, wherein the one or more processors are configured to execute the program instructions to cause the system to perform operations comprising: generating a first unique scan code; and associating the first unique scan code with the custom food item data record in the system database.

11. The system of claim 10, wherein the one or more processors are configured to execute the program instructions to cause the system to perform operations comprising: scanning the unique first scan code with the dietary client device; retrieving data of the custom food item data record from the system database; and displaying, via the GUI on the display screen of the dietary client device, the data of the custom food item data record.

12. The system of claim 8, wherein the one or more processors are configured to execute the program instructions to cause the system to perform operations comprising: receiving, via the vendor client device, user input having data describing one or more custom food items, wherein the data describing one or more custom food items includes nutrient content per serving data of the one or more custom food items; generating a custom dish data record in the system database having the data describing one or more custom food items and a number of servings of each of the one or more custom food items; and determining nutritional characteristics data of the custom dish data record.

13. The system of claim 12, wherein the one or more processors are configured to execute the program instructions to cause the system to perform operations comprising: generating a second unique scan code; and associating the second unique scan code with the custom dish data record in the system database.

14. The system of claim 13, wherein the one or more processors are configured to execute the program instructions to cause the system to perform operations comprising: scanning the second unique scan code with the dietary client device; retrieving data of the custom dish data record from the system database; and displaying, via the GUI on the display screen of the dietary client device, the data of the custom dish data record.

15. The system of claim 14, wherein the one or more processors are configured to execute the program instructions to cause the system to perform operations comprising: displaying, via the GUI on the display screen of the dietary client device, an output that describes how the nutritional characteristics data of the custom dish data record compares with recommended nutritional objectives data of the target diet data record.

16. The system of claim 15, wherein the output that describes how the nutritional characteristics data of the custom dish data record compares with recommended nutritional objectives data of the target diet data record comprises data describing how at least one nutritional characteristic of the custom dish data record does not meet at least one recommended nutritional objective of the target diet data record.

17. The system of claim 16, wherein the one or more processors are configured to execute the program instructions to cause data of a corrective dish modification to be displayed on the display screen of the dietary client device.

18. The system of claim 17, wherein the system database is stored in a data store of a system server, and wherein the dietary client device is one of a plurality of dietary client devices that are in network communication with the system server.

19. The system of claim 17, wherein the system database is stored in a data store of the dietary client device.

20. The system of claim 17, wherein the steps of: determining nutritional characteristics data of the custom dish data record; and displaying, via the GUI on the display screen of the dietary client device, an output that describes how the nutritional characteristic data of the custom dish data record compares with the data of the recommended nutritional objective data record of the target diet data record are both performed via a processor of the dietary client device.
